(19) United States
(12) Patent Application Publication
(10) Pub. No.: US 2015/0147761 A1
(43) Pub. Date: May 28, 2015

(54) SPECIFIC BIOMARKERS FOR HEPATOCELLULAR CARCINOMA (HCC)

(71) Applicants: Helmut E. Meyer, Recklinghausen (DE); Barbara Sitek, Witten (DE); Dominik A. Megger, Dortmund (DE); Thilo Bracht, Bochum (DE); Hideo Andreas Baba, Essen (DE); Joerg Friedrich Schlaak, Essen (DE); Frank Weber, Essen (DE)

(72) Inventors: Helmut E. Meyer, Recklinghausen (DE); Barbara Sitek, Witten (DE); Dominik A. Megger, Dortmund (DE); Thilo Bracht, Bochum (DE); Hideo Andreas Baba, Essen (DE); Joerg Friedrich Schlaak, Essen (DE); Frank Weber, Essen (DE)

(21) Appl. No.: 14/409,520

(22) PCT Filed: Jun. 20, 2013
(86) PCT No.: PCT/EP2013/062955

§ 371 Date: Dec. 19, 2014

(30) Foreign Application Priority Data
Jun. 20, 2012 (EP) 12172829.9

(51) Int. Cl. G01N 33/574; H01J 49/04; H01J 49/40; G01N 30/72; C07K 14/47; G01N 27/447

(52) U.S. Cl. CPC G01N 33/57438; C07K 14/47; G01N 27/447; H01J 49/40; G01N 30/7233; H01J 49/0418
USPC 435/7.1; 530/350; 436/501; 204/456; 250/282

(57) ABSTRACT

The invention relates to specific marker proteins (biomarkers) for Hepatocellular carcinoma (HCC). The invention relates to a method for the diagnostic study of biological samples of a human for Hepatocellular carcinoma, the sample being studied for one or more proteins as a marker for Hepatocellular carcinoma, a concentration of the proteins which is elevated or decreased in relation to the healthy state indicating the presence of Hepatocellular carcinoma, a diagnostic test kit and a method of screening compounds effective in HCC.

The invention relates to specific marker proteins (biomarkers) for Hepatocellular carcinoma (HCC). The invention relates to a method for the diagnostic study of biological samples of a human for Hepatocellular carcinoma, the sample being studied for one or more proteins as a marker for Hepatocellular carcinoma, a concentration of the proteins which is elevated or decreased in relation to the healthy state indicating the presence of Hepatocellular carcinoma, a diagnostic test kit and a method of screening compounds effective in HCC.

Hepatocellular carcinoma (HCC) currently is the fifth most common malignancy worldwide with an annual incidence up to 500 per 100000 individuals depending on the geographic region investigated. Whereas 80% of new cases occur in developing countries, the incidence increases in industrialized nations including Western Europe, Japan and the United States (El-Serag H B, N. Engl. J. Med. 1999; 340:745-750). To manage patients with HCC, tumor markers are very important tools for diagnosis, evaluation of disease progression, outcome prediction and evaluation of treatment efficacy. Several tumor markers have been reported for HCC, which include α-fetoprotein (AFP) (Di Bisceglie A M J Hepatol 2005), Lens culinaris agglutinin-reactive fraction of AFP (AFP-L3) (OKA H, J Gastroenteroll Hepatol 2001), and des-γ-carboxy prothrombin (DCP) (Liebman H A N Engl J Med 1984). However, none of these tumor markers show 100% sensitivity or specificity, which calls for new and better biomarkers.

In order to identify novel biomarkers of HCC, many clinical studies utilizing omics-based methods have been reported over the past decade. In particular, the proteomics-based approach has turned out to be a promising one, offering several quantification techniques to reveal differences in protein expression that are caused by a particular disease. In the most studies reported in literature, the well-established 2D-DIGE (two-dimensional difference in gel electrophoresis) technique has been applied for protein quantification followed by identification via mass spectrometry. Even if the quantification is very accurate and sensitive in this gel-based approach, the relatively high amount of protein sample necessary for protein identification is the major disadvantage of this technique. Several mass-spectrometry-based quantitative studies using labelling-techniques like SILAC (stable isotope labelling by amino acids in cell culture) or iTRAQ (isobaric tag for relative and absolute quantification) have been carried out as well for biomarker discovery of HCC. Here, the concomitant protein quantification and identification in a mass spectrometer allows high-throughput analyses. However, such experiments imply additional labelling reactions (in case of iTRAQ) or are limited to tissue culture systems (in case of SILAC). In the latter case, one can overcome the limitation by using the isotope-labelled proteins obtained from tissue culture as an internal standard added to a corresponding tissue sample. This approach is known as CDIT (culture-derived isotope tags) and was applied in a HCC study, very recently. Label-free proteomics based on quantification by ion-intensities or spectral counting offer another possibility for biomarker discovery. These approaches are cheap due to the lacking need of any labelling reagents and furthermore allow high-throughput and sensitive analyses in a mass spectrometer. A quantitative study of HCC using spectral counting has been reported, whereas an ion-intensity-based study has not been performed yet. Apart from these quantification strategies, protein alterations in HCC have been studied by MALDI imaging.

Proceeding from the described prior art, the object therefore presents itself of providing an improved method for studying biological samples for HCC, in which novel markers are used.

The object is achieved according to the invention by a method for studying biological samples of a human for HCC the sample being studied for one or more proteins as a marker for HCC, and an elevated level of the proteins indicating the presence of HCC, the proteins being selected from a group comprising proteins defined by SEQ ID No. 1 to 983 according to the enclosed sequence listening, isoforms of the proteins defined by SEQ ID No. 1 to 983, homologous of the proteins defined by SEQ ID NO. 1 to 983 and partial sequences of SEQ ID No. 1 to 983.

The present invention relates to a quantitative proteomic study characterized in a combination of two different techniques, namely the well-established 2D-DIGE (two-dimensional difference in gel electrophoresis) and a label-free ion-intensity-based quantification via mass spectrometry and liquid chromatography to identify HCC specific biomarkers. This is the first time such a combined study was performed with regard to hepatocellular carcinoma. By comparing the results of both studies high-confident biomarker candidates of HCC could be identified and 983 proteins were confirmed as specific biomarkers for HCC. Furthermore, the comparison demonstrates the complementarity of the gel- and LC-MS-based techniques. To verify the differential protein expressions detected in the proteomic studies underlying the present invention additional immunological validations of the identified specific biomarkers for HCC were performed.

The invention relates to a method for identifying biomarkers specific for a particular disease comprising the steps
a) determining if a particular protein is differentially expressed in cause of this particular disease by 2-D gel electrophoresis and
b) determining if this particular protein is differentially expressed in cause of this particular disease by liquid chromatography-mass spectrometry (LC-MS).

In one embodiment of the method the gel-based approach is SDS-Polyacrylamide gel electrophoresis, preferably 2D-DIGE.

In one embodiment of the method the LC-MS-based approach is a LC-MS-based label-free ion-intensity-based quantification, preferably MALDI, for example MALDI-TOF-MS or nan-HPLC-ESI-MS/MS.

In a preferred embodiment the invention relates to a method, wherein the gel-based approach is 2D-DIGE and wherein the LC-MS-based approach is MALDI, preferably MALDI-TOF-MS or nan-HPLC-ESI-MS/MS.

The present invention further relates to the use of the method for identifying biomarkers specific for a particular disease, to determine if a person has this particular disease, preferably to determine, if the person has HCC. In another preferred embodiment the present invention relates to a method, wherein the particular disease is hepatocellular carcinoma (HCC).

In one embodiment of the method the differential expression of the particular protein, the specific biomarker for HCC, is determined by comparing the amount of this protein in a biological sample of a person without the disease with the amount of this protein in a person with the disease.

In another preferred aspect the present invention relates to a biomarker for HCC identified by the method and selected from the proteins defined by SEQ ID No. 1 to 983, the respective homologes of SEQ ID No. 1 to 983 with at least 95% identity in amino acid sequence, the respective isoforms of proteins defined by SEQ ID No. 1 to 983, the respective partial sequences of SEQ ID No. 1 to 983.

In one embodiment the invention relates to a biomarker for HCC, characterized in that the biomarker is selected from PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31.

The invention relates to the use of one or more proteins selected from the proteins defined by SEQ ID No. 1 to 983, the respective homologes of SEQ ID No. 1 to 983 with at least 95% identity in amino acid sequence, the respective isoforms of proteins defined by SEQ ID No. 1 to 983, the respective partial sequences of SEQ ID No. 1 to 983 as biomarker(s) for hepatocellular carcinoma (HCC).

In one embodiment the invention relates to the use of one or more proteins, the specific biomarkers for HCC, wherein the protein(s) is/are selected from PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, homologous and partial sequences of these proteins as biomarker(s) for hepatocellular carcinoma (HCC).

In another embodiment the invention relates to the use of one or more proteins, the specific biomarkers for HCC, for differential diagnosis, in particular for early recognition, diagnosis, evaluation of disease progression, prediction of outcome, evaluation of treatment, surveillance of treatment of HCC.

The present invention further relates to a method for studying a biological sample for HCC, wherein the samples is studied for one or more biomarker(s) for HCC wherein the biomarker(s) is/are differentially expressed in relation to the healthy state indicating the presence of HCC, characterized in that the biomarker(s) is/are selected from the group comprising proteins defined by SEQ ID No. 1 to 983, the respective isoforms of the proteins defined by SEQ ID. No. 1 to 983, the respective homologues of SEQ ID No. 1 to 983 with at least 95% identity in amino acid sequence, the respective partial sequences of SEQ ID No. 1 to 983.

In one embodiment of the invention the method for studying a biological sample for HCC is characterized in that the biomarker(s) is/are selected from the group comprising proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, homologous and partial sequences of these proteins.

In one embodiment of the invention the method for studying a biological sample for HCC is characterized in that the sample is a human sample.

In one embodiment of the invention the method for studying a biological sample for HCC is characterized in that the sample is blood serum, blood plasma, whole blood, a biopsy sample, in particular a liver biopsy sample.

The present invention further relates to a diagnostic device or test kit for analysing the amount of at least one biomarker selected from the group comprising proteins defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences, and wherein the diagnostic device or test kit comprises detection reagents and further aids.

In one embodiment of the invention the diagnostic device or the test kit comprises a detection reagent that comprises an antibody specific for the respective biomarker.

The invention also relates to the above described uses, characterized in that at least two of the named biomarkers are used together, either simultaneously or sequentially.

The present invention further relates to the use of a method for identifying HCC specific biomarkers in a sample and wherein the HCC specific biomarkers are defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences.

The present invention further relates to the use of specific biomarkers for HCC selected from the group of specific biomarkers comprising the proteins defined by SEQ ID No. 1 to 983, preferably PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 the respective homologues with at least 95% identity in amino acid sequence, the respective isoforms, the respective partial sequences for screening pharmaceutical compounds for HCC.

The present invention further relates to a screening assay for the identification and validation of pharmaceutical compounds comprising one or more of the proteins selected from the group comprising the proteins defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences, and wherein the screening assay comprises detection reagents and further aids.

In the context of this invention, the term HCC comprises any form of Hepatocellular carcinoma (HCC). The terms are for example defined in Pschyrembel, Klinisches Wörterbuch [Clinical Dictionary], 263th edition, 2012, Berlin).

“Specific biomarkers for HCC”, “specific biomarkers” in the context of the invention are the proteins defined by SEQ ID No. 1 to 983 according to the sequence listening. Preferred biomarkers are the proteins listed in table 3. Specific biomarkers are also the respective isoforms, humongous and partial sequences of theses proteins. According to the invention also the nucleic acids e.g. RNA, DNA, cDNA encoding for the specific biomarkers are enclosed. Instead of the respective proteins or amino acids the respective nucleic acids encoding for these biomarkers could be used for early recognition, diagnosis, evaluation of disease progression, surveillance of treatment, or after treatment. In preferred embodiments of the invention the specific biomarker for HCC is a protein or peptide, e.g. one of the proteins SEQ ID No. 1-983, one of the proteins listed in Table 3, one of the proteins listed in Table 4 or a nucleic acid that encodes for one of those proteins.

An “Isoform” of the respective protein, the specific biomarker, is any of several different forms of the same protein. Different forms of a protein may be produced from related genes, or may arise from the same gene by alternative splicing. A large number of isoforms are caused by single-nucleotide-polymorphisms or SNPs, small genetic differences between alleles of the same gene. These occur at specific individual nucleotide positions within a gene. Isoforms comprise also proteins with the same or similar amino acid sequence but different post-translational modification, like glycosylation. A glycoform is an isoform of a protein that differs only with respect to the number or type of attached glycan. Glycoproteins often consist of a number of different glycoforms, with alterations in the attached saccharide or oligosaccharide.

A “Homologue” of the respective protein, the specific biomarker, is defined in terms of shared ancestry. Two segments of DNA can have shared ancestry because of either a speciation event (orthologs) or a duplication event (paralogs). The term “percent homology” and “sequence similarity” are used interchangeably. High sequence similarity might occur because of convergent evolution or because of chance. Such sequences are similar and are also included in the term according to the invention. Sequence regions that are homologous are also called conserved. Enclosed are also partial homology where a fraction of the sequences compared (are presumed to) share descent, while the rest does not. Many algorithms exist to cluster protein sequences into sequence families, which are sets of mutually homologous sequences, see for example databses HOVERGEN, HOMOLENS, HOGENOM. According to the invention homologues should display at least 80% or 90% or 95% identify in amino acid sequence, preferably 96% or 97%, most preferably 98% or 99% with one of the sequences SEQ ID NO. 1 to 983.

“Partial Sequences” according to the invention have for example at least 50% or 60%, preferably at least 70% or 80%, most preferred at least 90% or 95% of the amino acid sequence of SEQ ID No. 1 to 983.

The specific biomarkers for HCC may be identified as potential biomarkers during a proteome analysis of HCC in comparison to non-HCC tissue. For this purpose, liver biopsy samples were taken from patients having HCC.

The proteins were labelled using a pigment and subjected to a 2-D polyacrylamide gel electrophoresis using isoelectric focusing in the first dimension and SDS gel electrophoresis in the second dimension. The results were compared for HCC and non-HCC cells with the aid of software suitable for this purpose, to detect and quantify the spots which were amplified or decreased in the HCC sample in comparison to the non-HCC sample. The emission of the pigments, with which the proteins were labelled, was measured and analyzed.

“Difference gel electrophoresis” (DIGE) is a form of gel elektrophoresis where different protein samples can be labelled with fluorescent dyes (for example Cy3, Cy5, Cy2) prior to two-dimensional electrophoresis. Then, the labelled protein samples are mixed and put in the same gel. After the gel electrophoresis, the gel is scanned with the excitation wavelength of each dye one after the other, so each sample is analyzed separately. This technique is used to see changes in protein abundance like for example, between a sample of a healthy person and a sample of a person with HCC.

It overcomes limitations in traditional 2D electrophoresis that are due to inter-gel variation. This can be considerable even with identical samples. Since the proteins from the different sample types, e.g. healthy/diseased, virulent/non-virulent, are run on the same gel they can be directly compared. To do this with traditional 2D electrophoresis requires large numbers of time consuming repeats.

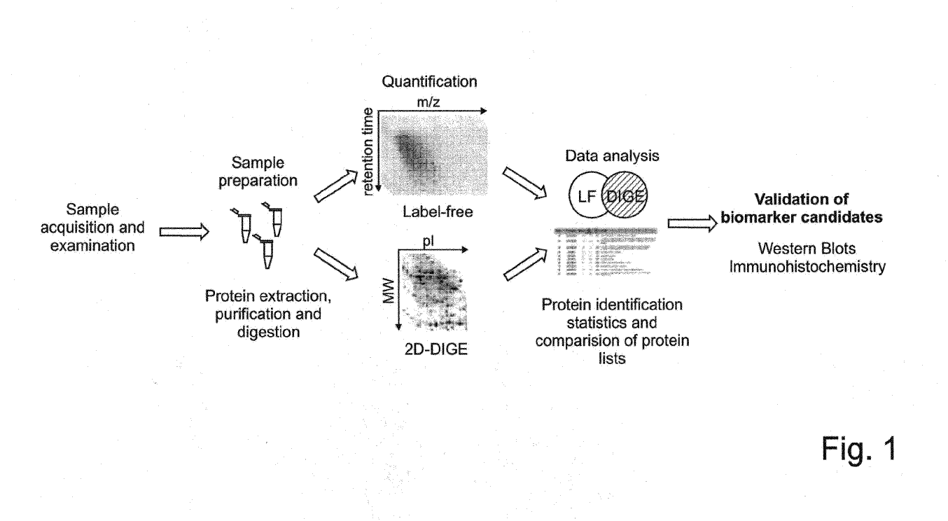

To identify novel biomarker candidates of hepatocellular carcinoma a study was performed that combines two complementary techniques of quantitative proteomics, namely the gel-based 2D-DIGE and the label-free LC-MS-based approaches. Following a straightforward workflow (FIG. 1), the differential protein expression in primary liver cancer tissue (n=7) in comparison to adjacent healthy liver tissue (n=7) was analyzed.

In the gel-based approach, a total of 1366 protein spots, represented in at least 70% of all investigated spot maps, were detected. Of these, only protein spots showing significant expression changes between healthy and malignant tissue specimens (p≦0.05 and 1.5-fold change of expression) have been isolated and analyzed. By the means of MALDI-MS and nano-LC-ESI-MS/MS analyses 240 proteins (148 non-redundant proteins) have been successfully identified. Among these, 55 proteins were found to be up- and 83 proteins down-regulated in HCC tumour tissue. Ten proteins showed variable regulation directions within several detected isoforms.

In the label-free approach, 31673 features comprising charges of 2+ or 3+ were detected. Significant differences in abundance between the two experimental groups were observed for 3507 of these features. Of these, 1038 regulated features have been assigned to peptide matches by the acquired tandem mass spectra. These identifications resulted in 476 significantly regulated proteins of which 284 were found to be up-regulated in tumour tissue and 194 down-regulated, respectively.

In summary, a total of 573 differentially expressed proteins were found, whereas 97 proteins were exclusively identified in the 2D-DIGE study and 425 proteins in the LC-MS study, respectively. Hence, only 57 differential proteins were identified irrespective of the applied quantification technique, which clearly shows that both approaches are complementary (Table 3). Except of eight proteins, the regulation directions of the proteins identified in both studies were equal. In four of the eight cases of inconsistent regulations, the protein expression already varies between several isoforms detected in the gel-based approach.

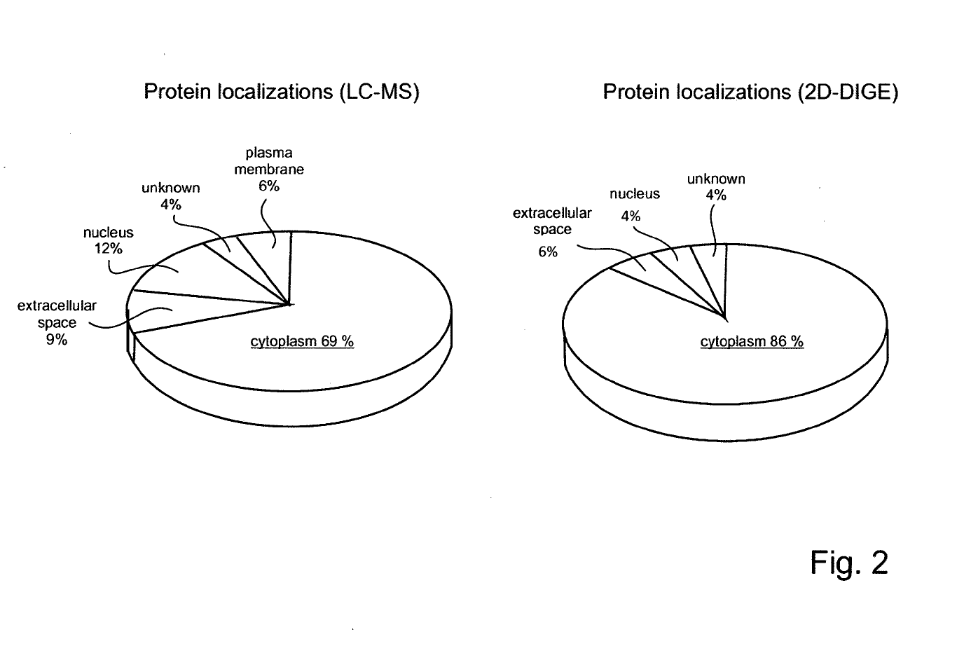

An analysis of the protein localizations revealed, that by using a gel-based approach mainly cytoplasmic proteins were detected, whereas the proteins detected in label-free approach widespread over a broader range of cellular localizations, in particular the plasma membrane (FIG. 2). Again, this clearly demonstrates the complementarily of both techniques.

TABLE 3
HCC specific biomarkers

| | IPI accession | | | | |
|---|---|---|---|---|---|
| | or Uniprot | | | | |
| | Accession | Gene | | Fold changes | |
| No | No. | name | Protein name | DIGE | LC-MS |
| 1 | IPI00015018 | PPA1 | Inorganic pyrophosphatase | 2 | 5.9 |
| 2 | IPI00448925 | IGHG1 | 44 kDa protein | 2.4 | 3.9 |
| | | IGHV4-31 | | | |
| 3 | IPI00553177 | SERPINA1 | Alpha-1-antitrypsin (isoform 1) | 2.7-3.7 | 3.6 |
| 4 | IPI00418471 | VIM | Vimentin | 3.1 | 2.9 |
| 5 | IPI00021405 | LMNA | Prelamin-A/C (isoform A) | 2.8-3.7 | 2.7 |
| 6 | IPI00554788 | KRT18 | Keratin, type I cytoskeletal 18 | 1.7 | 2.4 |
| 7 | IPI00219018 | GAPDH | Glyceraldehyde-3-phosphate dehydrogenase | 2.0-3.1 | 2.4 |
| 8 | IPI00479186 | PKM2 | Pyruvate kinase isozymes M1/M2 (isoform | 3.2 | 2.3 |
| | | | M2) | | |
| 9 | IPI00007765 | HSPA9 | HSPA9 Stress-70 protein, mitochondrial | 2.7-2.8 | 2.3 |
| 10 | IPI00003362 | HSPA5 | 78 kDa glucose-regulated protein | 3.8 | 2.2 |
| 11 | IPI00030275 | TRAP1 | Heat shock protein 75 kDa, mitochondrial | 3 | 2.2 |
| 12 | IPI00017855 | ACO2 | Aconitate hydratase, mitochondrial | 2.3-2.1 | 1.7 |
| 13 | IPI00003865 | HSPA8 | Heat shock cognate 71 kDa protein (isoform | 1.7-2.7 | 1.6 |
| | | | 1) | | |
| 14 | IPI00010720 | CCT5 | T-complex protein 1 subunit epsilon | 1.8 | 1.5 |
| 15 | IPI00011416 | ECH1 | Delta(3,5)-Delta(2,4)-dienoyl-CoA isomerase, | −2 | −1.5 |
| | | | mitochondrial | | |
| 16 | IPI00218733 | SOD1 | Superoxide dismutase [Cu—Zn] | −1.9 | −1.8 |
| 17 | IPI00218414 | CA2 | Carbonic anhydrase 2 | −2.3 | −1.8 |
| 18 | IPI00014439 | QDPR | Dihydropteridine reductase | −1.8 | −2.0 |
| 19 | IPI00009367 | AGXT | Serine--pyruvate aminotransferase | −2.3 | −2.2 |
| 20 | IPI00216057 | SORD | Sorbitol dehydrogenase | −2.4 | −2.3 |
| 21 | IPI00016801 | GLUD1 | Glutamate dehydrogenase 1, mitochondrial | −3.4-1.7 | −2.4 |
| 22 | IPI00889534 | CPS1 | Carbamoyl-phosphate synthase [ammonia], | −5.1-4.4 | −2.4 |
| | | | mitochondrial (isoform a precursor) | | |
| 23 | IPI00024990 | ALDH6A1 | Methylmalonate-semialdehyde | −3.5-2.1 | −2.4 |
| | | | dehydrogenase (acylating), mitochondrial | | |
| 24 | IPI00037448 | GRHPR | Glyoxylate reductase/hydroxypyruvate | −1.9 | −2.5 |
| | | | reductase | | |
| 25 | IPI00329331 | UGP2 | UTP-glucose-1-phosphate uridylyltransferase | −2 | −2.5 |
| | | | (isoform 1) | | |
| 26 | IPI00006663 | ALDH2 | Aldehyde dehydrogenase, mitochondrial | −2.5-2.4 | −2.6 |
| 27 | IPI00024993 | ECHS1 | Enoyl-CoA hydratase, mitochondrial | −3.5-2.2 | −2.7 |
| 28 | IPI00289524 | AKR1C4 | Aldo-keto reductase family 1 member C4 | −2.0 | −2.7 |
| 29 | IPI00218914 | ALDH1A1 | Retinal dehydrogenase 1 | −1.7 | −2.7 |
| 30 | IPI00165360 | MPST | 3-Mercaptopyruvate sulfurtransferase | −2.5 | −2.8 |
| 31 | IPI00020632 | ASS1 | Argininosuccinate synthase | −2.0-1.8 | −2.8 |
| 32 | IPI00027701 | ACADS | Short-chain specific acyl-CoA | −2.1 | −2.8 |
| | | | dehydrogenase, mitochondrial | | |
| 33 | IPI00218407 | ALDOB | Fructose-bisphosphate aldolase B | −3.5-2.4 | −3.0 |
| 34 | IPI00024623 | ACADSB | Short/branched chain specific acyl-CoA | −2.1-1.6 | −3.0 |
| | | | dehydrogenase, mitochondrial | | |
| 35 | IPI00216136 | KHK | Ketohexokinase (isoform C) | −1.7 | −3.2 |
| 36 | IPI00034308 | SARDH | Sarcosine dehydrogenase, mitochondrial | −3.3-2.8 | −3.3 |
| 37 | IPI00001441 | FTCD | Formimidoyltransferase-cyclodeaminase | −3.4 | −3.3 |
| | | | (isoform A) | | |
| 38 | IPI00010180 | CES1 | Liver carboxylesterase 1 (isoform 1) | −1.9 | −3.3 |
| 39 | IPI00025341 | BDH1 | D-beta-hydroxybutyrate dehydrogenase, | −2.4 | −3.5 |
| | | | mitochondrial | | |
| 40 | IPI00024896 | PBLD | Phenazine biosynthesis-like domain- | −2.8 | −3.6 |
| | | | containing protein | | |
| 41 | IPI00073772 | FBP1 | Fructose-1,6-bisphosphatase 1 | −2.3-1.8 | −4.0 |
| 42 | IPI00004101 | BHMT | Betaine-homocysteine S-methyltransferase 1 | −3.7-3.0 | −5.6 |
| 43 | IPI00215925 | GNMT | Glycine N-methyltransferase | −3.1 | −6.3 |
| 44 | IPI00745872 | ALB | Serum albumin (isoform 1) | −3.9-3.8 | 2.4 |
| 45 | IPI00419585 | PPIA | Peptidyl-prolyl cis-trans isomerase A | −2.9-1.7 | 1.7 |
| 46 | IPI00218342 | MTHFD1 | C-1-tetrahydrofolate synthase, cytoplasmic | 1.8 | −2.1 |
| 47 | IPI00030363 | ACAT1 | Acetyl-CoA acetyltransferase, mitochondrial | 1.8 | −2.3 |
| 48 | IPI00797038 | PCK2 | Phosphoenolpyruvate carboxykinase [GTP], | 2.1-3.3 | −2.9 |
| | | | mitochondrial (isoform 1) | | |
| 49 | IPI00032103 | GATM | Glycine amidinotransferase (isoform 1), | 1.8 | −3.2 |
| | | | mitochondrial | | |
| 50 | IPI00473031 | ADH1B | Alcohol dehydrogenase 1B | −6.3-3.1 | −3.4 |
| 51 | IPI00218899 | ADH4 | Alcohol dehydrogenase 4 (isoform 2) | −2.9-2.3 | −3.6 |
| 52 | IPI00186290 | eEF2 | Elongation factor 2 | | |
| 53 | O00418 | | Eucaryotic elongation factor 2 kinase | | |
| 54 | IPI00013890 | | ISOFORM 1 OF 14-3-3 PROTEIN SIGMA. | | |
| 55 | Q13043 | | serine/threonine kinase 4 | | |
| 56 | Q13188 | | serine/threonine kinase 3 (STE20 homolog) | | |
| 57 | Q9BXU1 | | serine/threonine kinase 31 | | |

The said “IPI accession” or “Uniprot Accession” of HCC specific biomarkers refers to Table 4 and correlated SEQ ID No.

### Selection of Biomarker Candidates for Further Validation

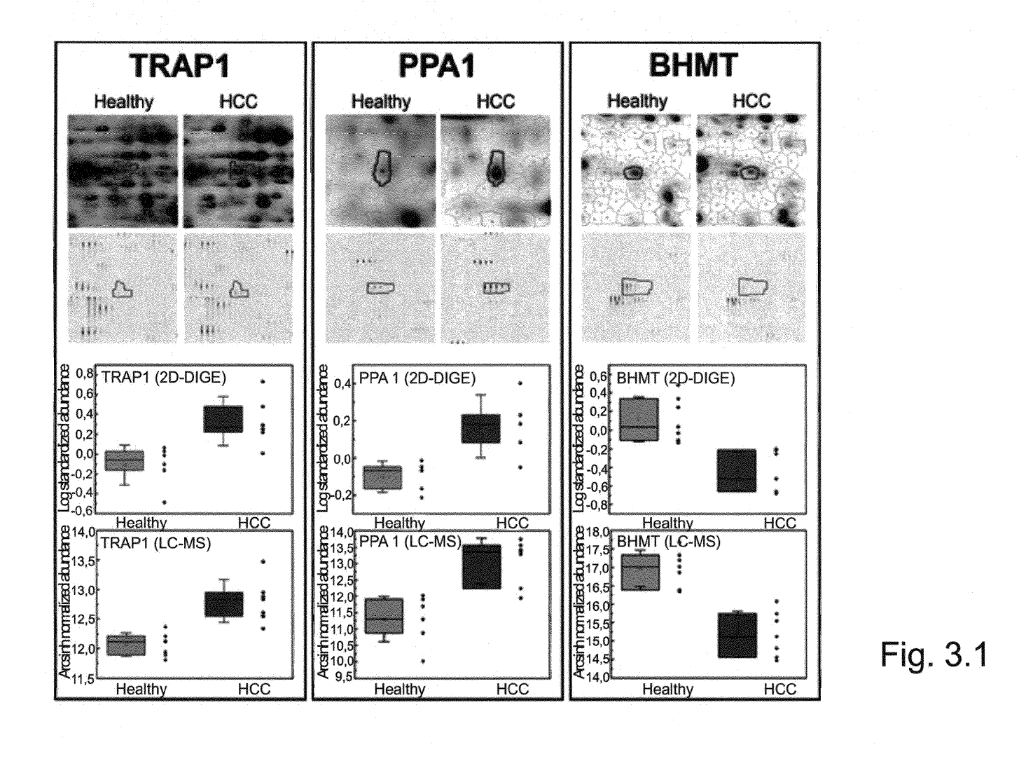

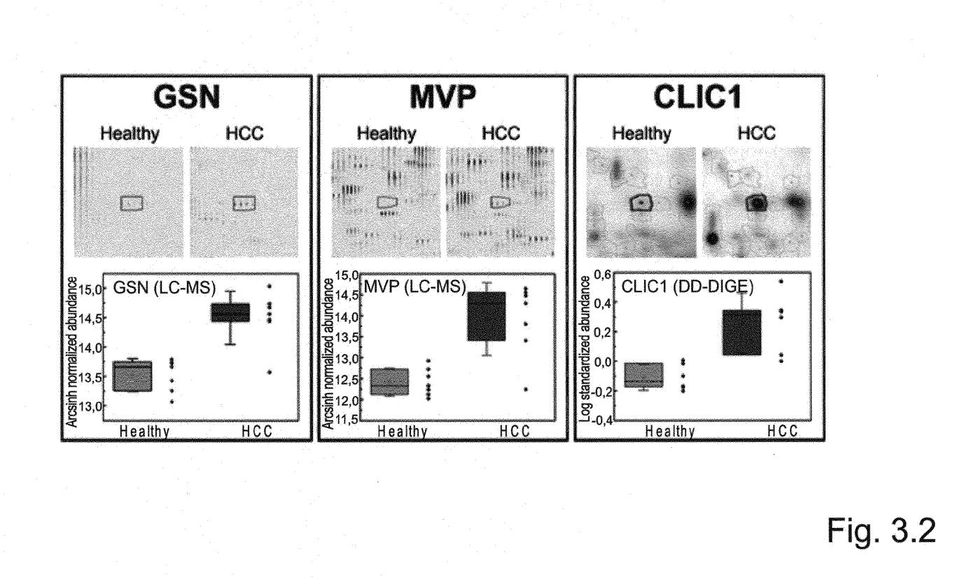

In order to verify the observed complementarities of the applied techniques and to identify biomarker candidates of HCC, for further validations several regulated proteins that were identified either in the 2D-DIGE study, the label-free study or the overlap of both were chosen. From the proteins exclusively identified in the gel-based 2D-DIGE approach the chloride intracellular channel protein 1 (CLIC1) was chosen, comprising a 2.5-fold over-expression in tumour tissue. From the complement of the label-free LC-MS based approach the major vault protein (MVP), which showed a 5.4-fold over-expression based on quantification with six unique peptides, as well as gelsolin (GSN) with a 2.8-fold higher expression (quantified with three unique peptides) was selected. The first regulated protein was chosen from the overlap of both studies is the tumour necrosis factor receptor-associated protein 1 (TRAP1), also known as heat shock protein 75 (HSP75). For this protein, fold changes of 3.0 and 2.2 were observed in the gel- and LC-MS-based approaches, respectively. As a second candidate from this group we selected inorganic pyrophosphatase 1 (PPA1), which was detected with fold changes of 2.0 in the 2D-DIGE experiment and 5.9 in the label-free approach. As an example for a biomarker candidate down-regulated in healthy tissue in comparison to HCC-tumour tissue betaine-homocysteine S-methyltransferase 1 (BHMT) was chosen for further validation. BHMT was found to be down-regulated in both studies with fold changes ranging from −3.0 to −3.7 in the gel-based approach and −5.6 in the label-free study (FIG. 3).

### Western Blotting and Immunohistochemistry

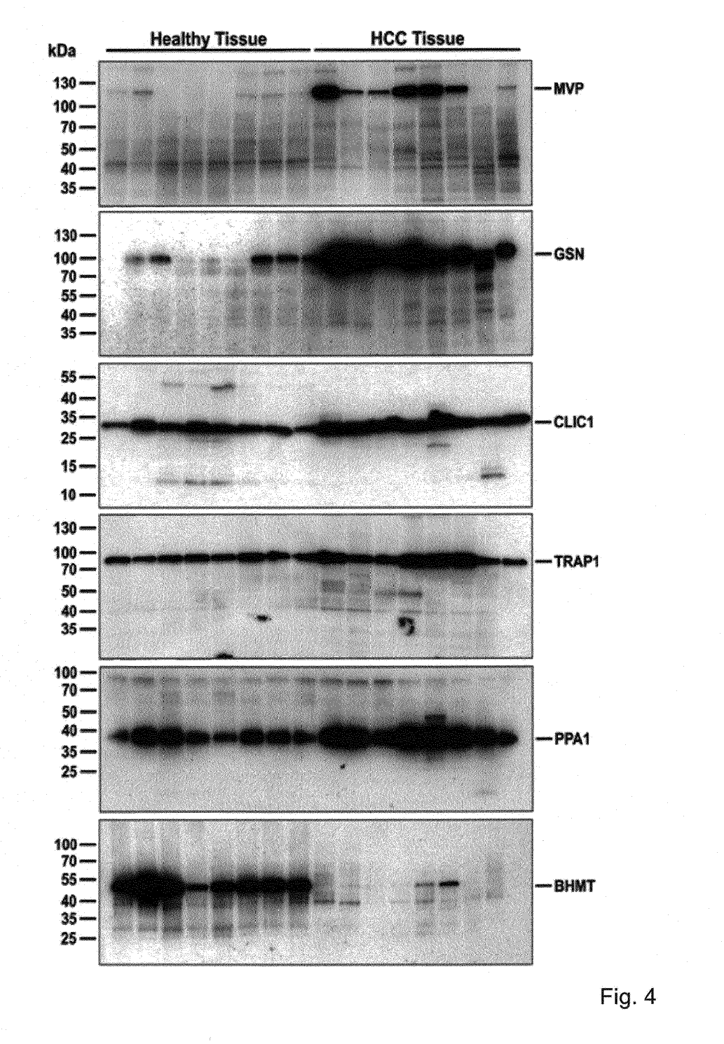

Biomarker candidates were investigated by western blot analysis of HCC-tissue (n=8) and healthy tissue (n=8), respectively. Analysis showed differential expression of all candidates in tumorous tissue in comparison to healthy tissue. MVP showed strong expression in six of eight tumour-samples whereas weak or no expression was observed in healthy tissue. Gelsolin was found with general high expression levels in HCC-tissue and only weak expression in healthy tissue. For CLIC1 enhanced expression levels were observed in all tumour samples. TRAP1 and PPA1 also showed higher expression levels in four of eight and five of eight HCC-tissue samples, respectively. For BHMT only little expression was detected in HCC-tissue in comparison to strong expression in all samples of healthy tissue (FIG. 4).

In addition to the western blot analysis immunohistochemical stainings of CLIC1, MVP, TRAP1 and PPA1 were done to validate these potential markers using an additional method. The normal liver showed CLIC1 positive non-hepatocytes but the hepatocytes were completely negative. In HCC the tumour cells displayed a strong positive signal in the cytoplasm and in the nuclei. In addition, the stroma cells were also positive for CLIC1. The antibody against MVP showed a immunoreactive signal in the cytoplasm of HCC cells but was negative in normal hepatocytes. TRAP1 was located in the cytoplasm of HCC cells but was negative in the non-tumour liver tissue. Using the antibody against pyrophosphatase 1 the tumour cells were slightly positive in the cytoplasm while the non-tumour liver cells were negative (data not shown).

In order to identify confident biomarker candidates of HCC and to elucidate the complementarities of the gel-based and LC-MS based quantification methods, the protein lists obtained in both studies were compared. Here, we observed a small overlap of only 57 proteins identified in both studies. This clearly shows the benefit of using different techniques in combination, which leads not only to an increased number of regulated proteins, that might act as disease markers or drug targets, but moreover makes candidates identified in both studies more confident. The latter assumption is clearly corroborated by the fact that the overlap includes several proteins that have already been associated to hepatocellular carcinoma and whose disease-related dysregulation has already been reported in numerous independent studies. However, the overlap also includes several proteins that were not associated to HCC earlier (e.g. TRAP1) and that are therefore new biomarkers of HCC.

In some cases, the comparison of protein regulations showed different results in the label-free and gel-based approach, respectively. However, in at least four of eight cases, this result is definitely caused by the detection of several up- or down-regulated isoforms of the same protein in the 2D-DIGE experiment. In such cases the regulations determined by the label-free bottom-up approach seem to be more reliable regarding the overall expression change of a protein. For example, the over-expressions of alcohol dehydrogenase 4 (ADH4) or peptidylprolyl isomerase A (PPIA) in HCC tissue specimens, as observed in the label-free approach, are in line with previously published data, whereas inconclusive results were obtained in the 2D-DIGE study.

In the current study an up-regulation of TRAP1 in hepatocellular carcinoma was found. TRAP1 is a member of the HSP90 family of molecular chaperones, which consists of three other major homologues, namely HSP90α, HSP90β and 94 kDa glucose-regulated protein (GRP94). In the present study, each of the four HSP90 homologues was found to be significantly over-expressed in cause of hepatocarcinogenesis, whereas only TRAP1 was identified irrespective of the applied quantification technique. For the homologues HSP90α, HSP90β and GRP94 the observed up-regulation has already been reported regarding several carcinoma types including HCC. However, the mitochondrial TRAP1 has not yet been investigated to such an extent. TRAP1 is involved in processes like drug resistance, cell survival, stress response, mitochondrial homeostatis and protein folding. Earlier, it was found to be over-expressed in colorectal (Landriscina, Cancer Lett., 2009) and nasopharyngeal carcinoma (Wang, Transl Med, 2008) as well as cisplatin-resistant ovarian cancer cells (Alvero, Cell Cycle, 2009; Esposito, Gynecologic oncology, 2010). In the prior case, the involvement of TRAP1 in drug-resistance was additionally studied by inhibiting TRAP1 activity with shepherdin (Landriscina, Cancer Lett., 2009) resulting in higher drug sensitivity. Hence, TRAP1 is not only a promising tumour marker candidate, for e.g. HCC, but moreover a potential drug target for improved cancer therapies, for e.g. HCC.

It was found that MVP is strongly up-regulated in hepatocellular carcinoma. The relatively large variance of expression levels observed in the label-free study and by western blotting is in line with previous observations and is most likely caused by an interindividual heterogeneity of MVP expression in liver tissue. Earlier, MVP has been found to be over-expressed in several human cancers such as pancreatic, breast, ovarian, urinary bladder carcinomas, melanomas, sarcomas and leukemias. However, in case of liver carcinomas a variable expression has been reported. MVP is the main constituent of the so called vaults, which are ribonucleoprotein particles with masses of approximately 13 MDa (Reference). Initially, vaults were supposed to be directly involved in the multidrug resistance of malignant tumours due to regulation of nuclear drug transport mechanisms. However, experiments with murine MVP knockout models showed no altered nuclear transport and chemoresistance. Recent observations suggest that vaults may be indirectly involved in drug resistance by modulation of cellular growth and survival signals. Here, interaction partners of MVP in the PI3K and MAPK pathway have been identified, suggesting that MVP might act as a regulatory protein in these signalling processes. More recently, MVP has been found to be involved in resistance to epidermal growth factor inhibition of several HCC-derived cell lines.

In the gel-based approach, chloride intracellular channel protein 1 (CLIC1) was found to be up-regulated in HCC tumour tissue. Members of CLIC protein family are widely expressed and involved in a variety of cellular processes like apoptosis, cell division or secretion. An HCC-related up-regulation of CLIC1 has already been reported in a proteomic study of hepatocellular carcinoma developed in patients with chronic hepatitis C infection as an underlying disease. Earlier, transcriptomics data were published that also revealed an over-expression of CLIC1 related to HCC which is in agreement with the present data. Within the patient cohort investigated in the study according to the invention, none of the patients had hepatitis B or C infections. Hence, the over-expression of CLIC1 in HCC seems to be irrespective of the underlying disease.

The ubiquitous, Ca²⁺-regulated actin-binding protein gelsolin (GSN) was also found to be over-expressed in tumorous tissue compared to adjacent healthy tissue. The protein exists in two major isoforms, namely the intracellular cytoplasmic one (cGSN) and a secreted form, also known as plasma gelsolin (pGSN). The three regulated peptides detected in the label-free approach are shared between those forms which makes a clear decision between both forms impossible at this point. Dysregulation of gelsolin in cause of several malignancies has been reported in numerous studies. In a high number of cancer types, including human breast, colorectal, gastric, bladder, lung, prostata, kidney, ovarian, pancreatic or oral cancers, gelsolin was down-regulated leading to the assumption that gelsolin might act as a tumour suppressor. However, in a subset of non-small cell lung cancers gelsolin was over-expressed. Furthermore, increased gelsolin levels have been associated to tumour recurrence and progression in urothelial tumours. The results from the label-free study and western blots according to the present invention show that GSN is also strongly up-regulated in HCC as well.

Inorganic pyrophosphatase (PPA1) was identified as a regulated protein in the label-free and 2D-DIGE approach. It catalyzes the hydrolysis of pyrophosphate to orthophosphate and is ubiquitously expressed. It has been shown to be differentially expressed in various types of cancer including enhanced expression in primary colorectal cancer (Tomonaga et al., 2004, Clin. Canc. Res.), lung adenocarcinoma (Chen et al., 2002, Clin. Canc. Res) and prostate cancer (Lexander H, 2005, Anal. Quant. Cytol. Histol.) and has also been shown to be expressed in a hepatocellular carcinoma cell line (Liang et al., 2002, J. of Chromatography B). However, in a proteomic pilot study of HCC in which tissue samples of only three patients have been analyzed using 2D gel electrophoresis, PPA1 has been found to be down-regulated (Matos et al., 2009, Journal of Surgical Research). In the present study, it was demonstrated with a larger cohort and two different quantification methods that PPA1 is significantly up-regulated in HCC. Furthermore, this result was validated using immunological methods. Thus PPA1 is also a diagnostic marker for HCC.

A strong decrease of BHMT expression in HCC tumour tissue has already been shown in gel-based proteomic studies (Liang et al., 2005, Proteomics; Sun et al., 2007, MCP) as well as on transcript level (Avila et al., 2000, J. of Hepatology). Very recently, the transcription of an aberrant splicing variant has been described as mechanism leading to decreased BHMT levels in HCC (Pellanda et al., 2012, Int. J. of Biochem. & Cell Biol.). BHMT is involved in homocysteine metabolism where it catalyzes the synthesis of methionine from betaine and homocysteine. Loss of BHMT function therefore leads to impaired hemostasis of 1-carbon metabolism and is directly associated with various diseases including hepatocellular carcinogenesis (Teng et al., 2011, JBC). In the present study the decreased expression of BHMT in HCC was confirmed for the first time using a label-free quantification method. BHMT expression was furthermore validated using western blot analysis as an example for a biomarker candidate down-regulated in HCC tumour tissue.

The following examples and figures are used to explain the invention without restricting the invention to the examples.

FIG. 1: Schematic representation of the applied workflow.

FIG. 2: Localizations of the differentially expressed proteins detected in the 2D-DIGE or LC-MS-based approach.

FIG. 3. Regulation patterns of selected proteins. Depending on the study in which the protein was detected, spot volume of the protein (2D-DIGE) and/or feature intensity of a representative peptide (LC-MS) in HCC and healthy samples are shown. Additionally, protein regulations within the investigated patient cohort are shown in the box plots (Boxes represent 25th and 75th percentile, whiskers indicate one standard deviation, the median is shown as black bar and the mean value as an empty square within box).

FIG. 4: Western blot of biomarker candidates.

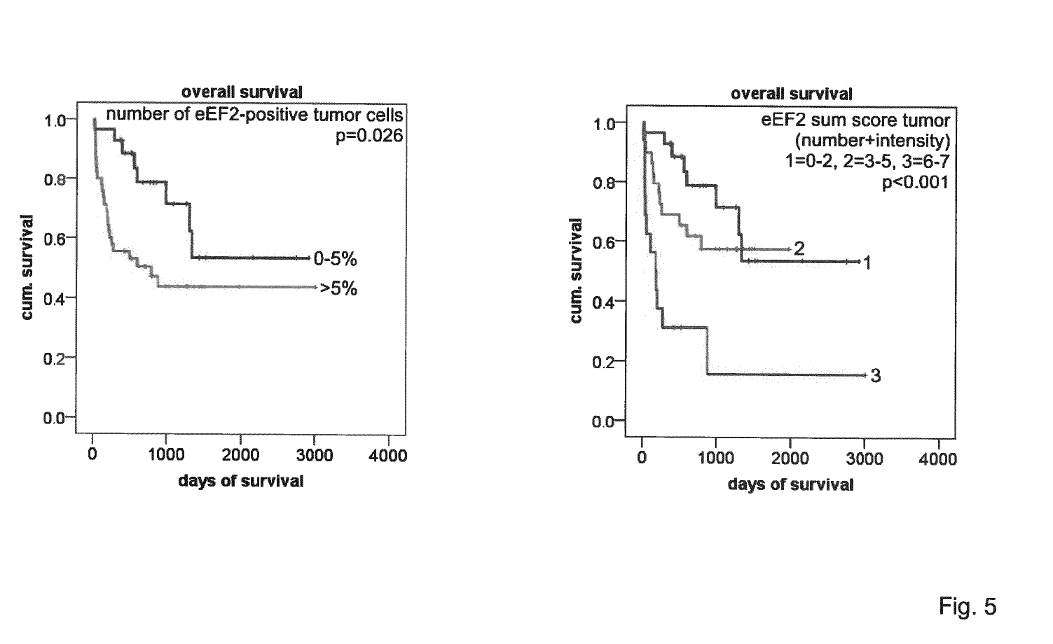

FIG. 5: Cummulated survival vs. survival with respect to eEF2 expression.

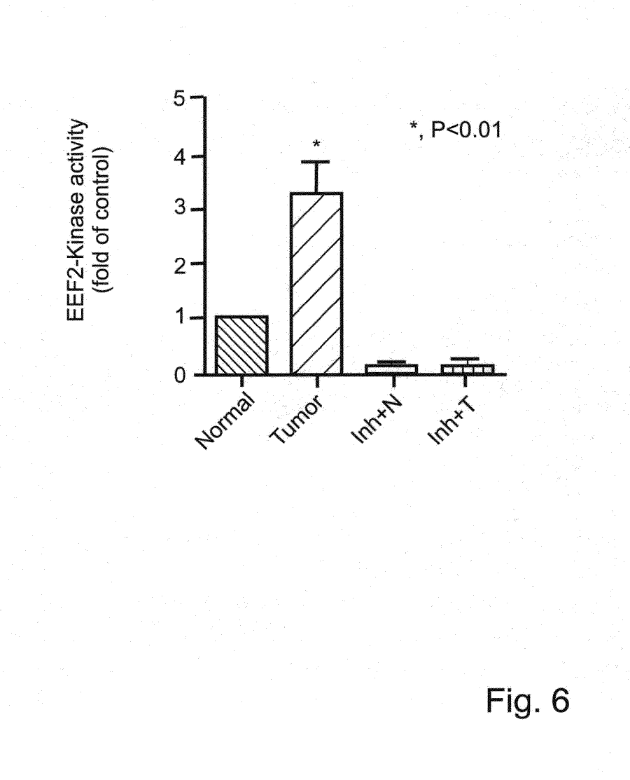

FIG. 6: eEF2-kinase activity in normal and HCC tissue.

### EXAMPLES

### Example 1

### Clinical Data

Tissue from hepatocellular carcinoma and non-tumour liver was collected from eight patients (four males and four females). The age of the patients ranged from 21 years to 76 years (mean 56.5). The tumours were classified according to the pathologic TNM (pTNM) system (seventh edition) (Sobin L H, Gospodarowicz M K, Wittekind C (2009) International union against cancer. TNM classification of malignant tumours, 7th edn. Wiley, New-York). All tumours except of one were classified as pT1, the tumor grading ranged from G1 to G3 and all tumours showed clear surgical margins. None of the patients had liver cirrhosis or hepatitis B or C infection. The patients and tumour characteristics are shown in table 1. Informed consent was obtained from every patient and the study protocol conforms to the ethical guidelines of the 1975 Declaration of Helsinki.

TABLE 1
Patient and tumour characteristics.

| ID | Gender | Age | T | N | G | V | R | Underlying liver disease |
|---|---|---|---|---|---|---|---|---|
| 1 | female | 57 | T1 | N0 | G3 | V0 | R0 | n.k. |
| 2 | female | 42 | T1 | NX | G2 | V0 | R0 | n.k. |
| 3 | male | 51 | T1 | N0 | G1 | V0 | R0 | n.k. |
| 4 | female | 21 | T2 | N1 | G2 | V1 | R0 | n.k. |
| 5 | male | 71 | T1 | NX | G3 | V0 | R0 | NASH |
| 6 | male | 65 | T1 | NX | G3 | V0 | R0 | NASH |
| 7^{a} | female | 69 | T1 | NX | G1 | V0 | R0 | n.k. |
| 8^{b} | male | 76 | T1 | NX | G1 | V0 | R0 | n.k. |

NASH = non alcoholic steatohepataitis.
n.k.= not known
NX = Regional lymph nodes cannot be assessed
^{a}From this patient, only tumour tissue was used in the proteomic study.
^{b}From this patient, only non-tumour tissue was used in the proteomic study.

### Example 2

### Tissue Preparation

Liver tumour and non-tumour tissue was collected and fixed in 4% buffered formalin, paraffin embedded and prepared for pathological examination and immunohistochemical evaluation. For the proteomics study the samples were immediately placed on ice, snap-frozen and stored at −80° C. The tissue samples were lysed by sonication (6×10 s pulses on ice) in sample buffer (30 mM TrisHCl; 2 M thiourea; 7 M urea; 4% CHAPS, pH 8.5). After centrifugation at 15.000 g for 5 min, the supernatant was collected and protein concentration was determined using the Bio-Rad Protein Assay (Bio-Rad, Hercules, Calif.).

### Example 3

### 2D-DIGE Analysis

### Example 3.1

### Protein Labelling

Proteins were labelled using cyanine dyes in the ratio 50 μg protein to 400 pmol dyes (minimal labelling dyes, GE Healthcare). The labelling reaction was performed according to the manufacturer's instructions. Samples of HCC-tissue and healthy tissue were randomized by labelling with Cy3 dye or Cy5 dye to avoid any dye biases. The internal standard, which is a mixture of same amounts of all analyzed samples, was labelled with Cy2 dye.

### Example 3.2

### 2D Electrophoresis

The seven sample mixtures, including appropriate Cy3- and Cy5-labeled pairs and a Cy2-labeled internal standard, were generated and per 100 μl cell lysate, 10 μl DTT (1.08 g/ml; BioRad) and 10 μl Ampholine 2-4 (Amersham Biosciences) were added. IEF was performed using tube gels (20 cm×0.9 mm) containing carrier ampholytes (CA-IEF) and applying a voltage gradient in an IEF-chamber produced in house. After IEF, the ejected tube gels were incubated in equilibration buffer (125 mM Tris, 40% (w/v) glycerol, 3% (w/v) SDS, 65 mM DTT, pH 6.8) for 10 min. The second dimension (SDS-PAGE) was performed on (15.2% total acrylamide, 1.3% bisacrylamide) polyacrylamide gels using a Desaphor VA 300 system. IEF tube gels were placed onto the polyacrylamide gels (20 cm×30 cm×0.7 mm) and fixed using 1.0% (w/v) agarose containing 0.01% (w/v) bromphenol blue dye (Riedel de-Haen, Seelze, Germany). For identification of proteins by MS, 250 μg total protein was applied to IEF tube gels (20 cm×1.5 mm) and subsequently to preparative SDS-PAGE gels (20 cm×30 cm×1.5 mm). Silver post-staining was performed after gel scanning using a MS-compatible protocol as described elsewhere.

### Example 3.3

### Scanning, Image Analysis and Statistics

SDS-PAGE gels were scanned using a Typhoon 9400 scanner (Amersham Biosciences). Excitation and emission wavelengths were chosen specifically for each of the dyes according to recommendations of the manufacturer. Images were pre-processed using the ImageQuant™ software (GE Healthcare). Intra-gel spot detection, inter-gel matching and normalization of spot intensities were performed using the Differential In-gel Analysis (DIA) mode and Biological Variation Analysis (BVA) mode of DeCyder 2D™ software (GE Healthcare), respectively. Spot intensities were normalized to the internal standard. The Extended Data Analysis tool (EDA), implemented in the DeCyder 2D™ software package, was used for the statistical analysis of the 2D-DIGE experiments. Here, only spots appearing in at least 70% of all analyzed and matched spot maps were chosen for further analysis. Significantly regulated proteins were identified by Student's t-test including a false-discovery-rate correction. Protein spots differentially expressed (p≦0.05, Av. Ratio 1.5) between HCC and healthy samples were identified using MALDI-TOF-MS or nano-HPLC-ESI-MS/MS.

### Example 3.4

### Digestion and Protein Identification

In-gel digestion of proteins was performed with trypsin following standard protocols and the obtained peptides were extracted from the gel matrix. MALDI-TOF-MS analyses were performed on an UltraFlex™ II instrument (Bruker Daltonics). For nano-HPLC-ESI-MS/MS experiments an Ultimate 3000 RSLCnano system online coupled to a Bruker Daltonics HCT plus ion trap instrument equipped with a nanoelectrospray ion source (Bruker Daltonics) was used. For protein identification database searches against the IPI human database were performed using Mascot. Further details regarding the experimental setup, search parameters or identification threshold were described earlier.

### Example 4

### Label-Free Analysis

### Example 4.1

### In-Gel Digestion and Sample Preparation

Prior to LC-MS analysis, 5 μg of each protein sample were loaded on a 4-20% SDS-PAGE gel (Anamed) and allowed to run into the gel for about 1 cm (15 min at 50 V). After Coomassie-staining, in-gel trypsin digestion was performed following standard procedures. The generated peptides were extracted by sonication (15 min, ice cooling) of the gel pieces in approximately 20 μl of 50% acetonitrile in 0.1% TFA, twice. Afterwards, acetonitrile was removed by vacuum centrifugation and peptide concentration of the resulting solution was determined by amino acid analysis performed on an ACQUITY-UPLC with an AccQ Tag Ultra-UPLC column (Waters, Eschborn, Germany) calibrated with Pierce Amino Acid Standard (Thermo Scientific, Bremen, Germany). Prior to LC-MS analysis, samples were diluted with 0.1% TFA to adjust a peptide concentration of 23.3 ng/μl.

### Example 4.2

### LC-MS/MS Analysis

Quantitative label-free analyses were performed on an Ultimate 3000 RSLCnano system (Dionex) online coupled to a LTQ Orbitrap Velos instrument (Thermo Scientific, Bremen, Germany). For each analysis 15 μl of sample were injected, corresponding to an amount of 350 ng tryptic digested proteins. The peptides were preconcentrated with 0.1% TFA on a trap column at a flow rate of 7 μl/min for 10 min. Subsequently, the peptides were transferred to the analytical column and separated using a xxx_min gradient from 5-40% solvent B at a flow rate of 300 nl/min (solvent A: 0.1% formic acid, solvent B: 0.1% FA 84% acetonitrile). The column oven temperature was set to 60° C. The mass spectrometer was operated in a data-dependent mode. Full scan MS spectra were acquired at a mass resolution of 30000 in the Orbitrap analyzer. Tandem mass spectra of the twenty most abundant peaks were acquired in the linear ion trap by peptide fragmentation using collision-induced dissociation.

### Example 4.3

### Peptide Quantification and Filtering

Progenesis LC-MS™ software (version, Nonlinear) was used for the ion-intensity-based label-free quantification. After importing the .raw files, one sample was selected as a reference run to which the retention times of the precursor masses in all other samples were aligned to. In the following, a list of features was generated including the m/z values of all eluted peptides at given retention times. For further analysis, only features comprising charges of 2+ and 3+ were selected. Subsequently, the raw abundances of each feature were automatically normalized for correcting experimental variations. The detailed procedure of normalization is described elsewhere. In a following step, the samples were grouped corresponding to the selected experimental design, in this case a two-group comparison between “healthy” and “HCC”. Differences of peptide abundances between both groups were assigned to be significant if the following filter criteria were satisfied (ANOVA p-value ≦0.05 and q-value ≦0.05) in the following statistical analysis. Due to the fact, that multiple MS/MS spectra were acquired for the same features, only the fragment-ion spectra of the ten most intense precursors of a feature were selected for generation of peak list exported to a Mascot generic file.

### Example 4.4

### Protein Identification

The generated .mgf file was searched against the IPI human database using Mascot. The following search parameters were applied: variable modifications propionamide (C) and oxidation (M), tryptic digestion with up to one missed cleavage, #¹³C=1, precursor ion mass tolerance of 5 ppm and fragment ion mass tolerance of 0.4 Da. For further analysis, only peptides with mascot ion scores >37 (p≦0.01 identity threshold) were chosen. By importing the list of identified peptides in Progenesis LC-MS, the previously quantified features were matched to the corresponding peptides.

### Example 4.5

### Protein Quantification and Filtering

For the protein quantification, only non-conflicting peptides were chosen and the protein-grouping function implemented in Progenesis LC-MS was disabled. However, conflicting peptides matching to more than one protein hit were used for protein identification in order to make them more confident. At the protein level, the significance of expression changes was again tested by calculating an ANOVA p-value and a q-value. Proteins not satisfying the significance criteria (ANOVA p-value ≦0.05 and q-value ≦0.05) were filtered out. Finally, proteins showing less than 1.5-fold change of expression were discarded as well.

### Example 5

### Analysis of Regulated Proteins

The Ingenuity Pathway Analysis software (Version 12402621, Ingenuity Systems, www.ingenuity.com) was used to assign the localizations of the regulated proteins detected in the label-free and 2D-DIGE experiment.

### Example 6

### Western Blotting

Protein concentration was determined by amino acid analysis. Equal amounts of 15 μg protein per sample were separated by SDS-PAGE on a 4%-20% polyacrylamide gel (Criterion TGX, Bio-Rad, Hercules, USA). Proteins were subsequently transferred onto nitrocellulose membrane (Trans-Blot Turbo, Bio-Rad, Hercules, USA) and membranes were blocked with StartingBlock blocking buffer (Thermo Scientific, Bremen, Germany) for one hour at room temperature. First antibodies anti-CLIC1 (Clone 2D4, Abnova, Heidelberg, Germany, dilution 1:1000), anti-MVP (Clone 1032, Acris, Herford, Germany, dilution 1:1000), anti-PPA1 (ab96099, abcam, Cambridge, UK, dilution 1:5000), anti-TRAP1 (clone EPR5381, abcam, Cambridge, UK, dilution 1:15000), anti-GSN (clone GS-2C4, Sigma-Aldrich, Munich, Germany, dilution 1:1000) and anti-BHMT (clone EPR6782, Epitomics, Burlingame, USA, dilution 1:20000) were diluted in StartingBlock and incubated with membranes over night at 4° C. Horseradish peroxidase-labeled secondary antibodies (Jackson ImmunoResearch, Newmarket, UK) were used for detection for one hour at room temperature. Bound antibodies were visualized by enhanced chemoluminescence and exposure to hyperfilm (GE Healthcare, Munich, Germany).

### Example 7

### Immunohistochemistry

Paraffin embedded 4 um slides were dewaxed and pre-treated in EDTA buffer (pH 9) at 95° C. for 30 min. All Immunohistochemical stains of were performed with an automated staining device (Dako Autostainer, Glostrup, Denmark). Both, the source of the primary antibodies and the technical staining details of the automatically performed stainings are listed in table 2. All stains were developed using a Polymer Kit (ZytoChemPlus (HRP), POLHRS-100, Zytomed Systems). Replacement of the various primary antibodies by mouse or rabbit immunoglobulin served as negative controls.

Immunohistochemical staining was made of HCC and the corresponding non-tumour liver from the same patient. CLIC1: Immunohistochemistry against CLIC1 shows reactivity in sinusoidal lining cells but shows no signal in hepatocytes. In HCC strong reactivity is present in the cytoplasm and nuclei of tumour cells and also in non-tumour stroma cells. MVP: In the normal liver MVP is located in some nucleated blood cells but hepatocytes are negative in contrast to HCC with positive signals in the cytoplasm of tumour cells. TRAP1: Immunohistochemistry against TRAP1 shows strong reactivity in HCC cells, but is negative in the normal liver. PPA1: The antibody against PPA1 shows a faint reactivity in HCC cells, but is completely negative in the normal liver (results not shown).

TABLE 2
Antibodies used for immunohistochemistry.

| Antibody | Distributor | Code | Source | AB concentration |
|---|---|---|---|---|
| TRAP 1 | abcam | ab109323 | Rabbit monoclonal | 1:200, 30 min. RT |
| LRP/MVP | Kamiya | MC-603 | Mouse monoclonal | 1:100, 30 min. RT |
| Pyrophosphatase-1 | abcam | ab96099 | Rabbit polyclonal | 1:500, 30 min. RT |
| CLIC1 | Abnova | H00001192-M01 | Mouse monoclonal | 1:9000, 30 min. RT |

AB:antibody
RT:room temperature

### Example 8

### eEF2 as Specific Biomarker for HCC

In validation experiments using immunhistochemistry the protein eEF2 was able to discriminate non-HCC-tissue from HCC-tissue obtained from 78 patients. The difference was significant. In addition, eEF2 distinguishes between patients with favourable prognosis and patients with unfavourable prognosis. These results were significant (n=75). FIG. 5 (left part) shows that patients with HCC and no or only little eEF immuno-expression (score 0.1) survive significant longer than patients with HCC that have many eEF-positive cells within the tumour (score 2.3). If the intensity of the staining is also taken into account when evaluating the immunhistochemical data, patients can be identified, that have a very bad prognosis. Patients with a very bad prognosis have many eEF2 positive cells within the tumour and strong reactivity (strong intensity of the staining; p<0.0001). This is shown in the right part of FIG. 5.

### Example 9

### eEF2-Kinase-Phosphorylation Assay

In addition, the kinase of eEF2 was investigated using 7 tissues from HCC patients and 7 control tissues.

Lysates from liver tissue were prepared using lysis buffer (0.5% (v/v) NP-40, 150 mM NaCl, 1 mM CaCl₂, 25 mM Na4P2O7, 50 mM β-glycerol phosphate disodium salt, 2 mM EDTA, 2 mM EGTA, 25 mM Tris, pH 8.0, 10% (v/v) glycerol, 10 μg ml⁻¹ soybean trypsin inhibitor, 1 mM benzamidine, 1 mM PMSF, 50 mM NaF, 0.1 mM Na3VO4, 0.002% (w/v) NaN3). eEF2-Kinase was immunoprecipitated using eEF2K antibodies (#3692, Cell Signaling; 5 ml/1 mg lysate) bound to Protein A sepharose beads and with gentle rotation for 2 h at 4° C. Beads were washed three to four times in phosphorylation buffer containing 50 mM Hepes (pH7.4), 10 mM MgCl₂ and 1 mM CaCl₂. For the kinase assay His-tagged eEF2 protein (eEF2 fragment corresponding to amino acids 9-165; Abcam 91684; 0.5 μg/sample), Calmodulin (2.5 mg/sample; Sigma, C4874), 10 μM ATP and [γ-³²P]ATP (0.5-0.75 μCi/sample; Fa. Hartmann) were added to immunoprecipiptated eEF2 kinase. Unspecific kinase activity was determined by addition of the eEF2 kinase inhibitor NH125 (3 μM, Calbiochem) to indicated samples. After 20 min at 30° C., the reaction was stopped by the addition of Laemmli buffer. Proteins were separated by SDS-PAGE and phosphorylation of His-eEF2 was detected and quantified by PhosphorImager analysis. Protein levels/amounts of immunoprecipitated eEF2K were controlled by Western blot analysis.

A significant difference of eEF2-kinase activity was determined between HCC and non-HCC tissue (FIG. 6).

### Example 10

### Serine/Threonine Kinase 3 and 4

Serine/threonine kinase 3 and 4 were also identified as a marker for HCC by using tumour tissue from 11 patients with HCC and 11 tissues from controls. These proteins were validated by immunhistochemical approach using tumour tissue from 290 patients. Serine/threonine kinase 3 and 4 are suitable as a diagnostic and prognostic marker for HCC.

TABLE 4
HCC specific biomarkers / proteins

| SEQ | IPI Accession | |
|---|---|---|
| ID | or Uniprot | |
| No. | Accession No. | Protein Name |
| 1 | IPI00010290 | FABP1 PROTEIN (FRAGMENT). |
| 2 | IPI00218896 | ALCOHOL DEHYDROGENASE 1A. |
| 3 | IPI00473031 | ALCOHOL DEHYDROGENASE 1B. |
| 4 | IPI00465343 | ALCOHOL DEHYDROGENASE 1C. |
| 5 | IPI00218407 | FRUCTOSE-BISPHOSPHATE ALDOLASE B. |
| 6 | IPI00410714 | HEMOGLOBIN SUBUNIT ALPHA. |
| 7 | IPI00011062 | ISOFORM 1 OF CARBAMOYL-PHOSPHATE SYNTHASE [AMMONIA], |
| | | MITOCHONDRIAL. |
| 8 | IPI00465439 | FRUCTOSE-BISPHOSPHATE ALDOLASE A. |
| 9 | IPI00006663 | ALDEHYDE DEHYDROGENASE, MITOCHONDRIAL. |
| 10 | IPI00218914 | RETINAL DEHYDROGENASE 1. |
| 11 | IPI00103467 | ALDEHYDE DEHYDROGENASE X, MITOCHONDRIAL. |
| 12 | IPI00218899 | ISOFORM 2 OF ALCOHOL DEHYDROGENASE 4. |
| 13 | IPI00158280 | FORMYLTETRAHYDROFOLATE DEHYDROGENASE ISOFORM A VARIANT. |
| 14 | IPI00448095 | L-XYLULOSE REDUCTASE. |
| 15 | IPI00004101 | BETAINE--HOMOCYSTEINE S-METHYLTRANSFERASE 1. |
| 16 | IPI00218733 | SUPEROXIDE DISMUTASE [CU—ZN]. |
| 17 | IPI00008934 | HYDROXYMETHYLGLUTARYL-COA SYNTHASE, MITOCHONDRIAL. |
| 18 | IPI00008475 | HYDROXYMETHYLGLUTARYL-COA SYNTHASE, CYTOPLASMIC. |
| 19 | IPI00554648 | KERATIN, TYPE II CYTOSKELETAL 8. |
| 20 | IPI00219446 | PHOSPHATIDYLETHANOLAMINE-BINDING PROTEIN 1. |
| 21 | IPI00016801 | GLUTAMATE DEHYDROGENASE 1, MITOCHONDRIAL. |
| 22 | IPI00010180 | ISOFORM 1 OF LIVER CARBOXYLESTERASE 1. |
| 23 | IPI00018278 | HISTONE H2A.V. |
| 24 | IPI00414676 | HEAT SHOCK PROTEIN HSP 90-BETA. |
| 25 | IPI00027230 | ENDOPLASMIN. |
| 26 | IPI00003865 | ISOFORM 1 OF HEAT SHOCK COGNATE 71 KDA PROTEIN. |
| 27 | IPI00003362 | 78 KDA GLUCOSE-REGULATED PROTEIN. |
| 28 | IPI00001539 | 3-KETOACYL-COA THIOLASE, MITOCHONDRIAL. |
| 29 | IPI00022793 | TRIFUNCTIONAL ENZYME SUBUNIT BETA, MITOCHONDRIAL. |
| 30 | IPI00020632 | ARGININOSUCCINATE SYNTHASE. |
| 31 | IPI00005038 | RIBONUCLEASE UK114. |
| 32 | IPI00456429 | UBIQUITIN-60S RIBOSOMAL PROTEIN L40. |
| 33 | IPI00382470 | ISOFORM 2 OF HEAT SHOCK PROTEIN HSP 90-ALPHA. |
| 34 | IPI00329331 | ISOFORM 1 OF UTP--GLUCOSE-1-PHOSPHATE URIDYLYLTRANSFERASE. |
| 35 | IPI00024993 | ENOYL-COA HYDRATASE, MITOCHONDRIAL. |
| 36 | IPI00216057 | SORBITOL DEHYDROGENASE. |
| 37 | IPI00018206 | ASPARTATE AMINOTRANSFERASE, MITOCHONDRIAL. |
| 38 | IPI01014563 | FERRITIN LIGHT CHAIN. |
| 39 | IPI00291560 | ISOFORM 1 OF ARGINASE-1. |
| 40 | IPI00784154 | 60 KDA HEAT SHOCK PROTEIN, MITOCHONDRIAL. |
| 41 | IPI00182933 | ISOFORM 2 OF CYTOCHROME B5. |
| 42 | IPI00220362 | 10 KDA HEAT SHOCK PROTEIN, MITOCHONDRIAL. |
| 43 | IPI00025252 | PROTEIN DISULFIDE-ISOMERASE A3. |
| 44 | IPI00019912 | PEROXISOMAL MULTIFUNCTIONAL ENZYME TYPE 2. |
| 45 | IPI00303476 | ATP SYNTHASE SUBUNIT BETA, MITOCHONDRIAL. |
| 46 | IPI00465436 | CATALASE. |
| 47 | IPI00073772 | FRUCTOSE-1,6-BISPHOSPHATASE 1. |
| 48 | IPI00217871 | DELTA-1-PYRROLINE-5-CARBOXYLATE DEHYDROGENASE, MITOCHONDRIAL. |
| 49 | IPI00797038 | ISOFORM 1 OF PHOSPHOENOLPYRUVATE CARBOXYKINASE [GTP], |
| | | MITOCHONDRIAL. |
| 50 | IPI00218297 | 4-HYDROXYPHENYLPYRUVATE DIOXYGENASE. |
| 51 | IPI00020955 | 3-OXO-5-BETA-STEROID 4-DEHYDROGENASE. |
| 52 | IPI00179709 | ISOFORM 1 OF TUBULIN ALPHA-3C/D CHAIN. |
| 53 | IPI00037448 | GLYOXYLATE REDUCTASE/HYDROXYPYRUVATE REDUCTASE. |
| 54 | IPI00217966 | ISOFORM 1 OF L-LACTATE DEHYDROGENASE A CHAIN. |
| 55 | IPI00022891 | ADP/ATP TRANSLOCASE 1. |
| 56 | IPI00029784 | UDP-GLUCURONOSYLTRANSFERASE 2B7. |
| 57 | IPI00031708 | FUMARYLACETOACETASE. |
| 58 | IPI00012728 | ISOFORM 1 OF LONG-CHAIN-FATTY-ACID--COA LIGASE 1. |
| 59 | IPI00216308 | VOLTAGE-DEPENDENT ANION-SELECTIVE CHANNEL PROTEIN 1. |
| 60 | IPI00012303 | ISOFORM 1 OF SELENIUM-BINDING PROTEIN 1. |
| 61 | IPI00645452 | UNCHARACTERIZED PROTEIN. |
| 62 | IPI00216133 | BILE SALT SULFOTRANSFERASE. |
| 63 | IPI00025512 | HEAT SHOCK PROTEIN BETA-1. |
| 64 | IPI00009904 | PROTEIN DISULFIDE-ISOMERASE A4. |
| 65 | IPI00024990 | METHYLMALONATE-SEMIALDEHYDE DEHYDROGENASE [ACYLATING], |
| | | MITOCHONDRIAL. |
| 66 | IPI00893541 | 14 KDA PROTEIN. |
| 67 | IPI00419585 | PEPTIDYL-PROLYL CIS-TRANS ISOMERASE A. |
| 68 | IPI00418169 | ISOFORM 2 OF ANNEXIN A2. |
| 69 | IPI00001441 | ISOFORM A OF FORMIMIDOYLTRANSFERASE-CYCLODEAMINASE. |
| 70 | IPI00329033 | DIMETHYLANILINE MONOOXYGENASE [N-OXIDE-FORMING] 3. |
| 71 | IPI00646304 | PEPTIDYL-PROLYL CIS-TRANS ISOMERASE B. |
| 72 | IPI00031522 | TRIFUNCTIONAL ENZYME SUBUNIT ALPHA, MITOCHONDRIAL. |
| 73 | IPI00218414 | CARBONIC ANHYDRASE 2. |
| 74 | IPI00296645 | MICROSOMAL TRIGLYCERIDE TRANSFER PROTEIN LARGE SUBUNIT. |
| 75 | IPI00396378 | ISOFORM B1 OF HETEROGENEOUS NUCLEAR RIBONUCLEOPROTEINS A2/B1. |
| 76 | IPI00010796 | PROTEIN DISULFIDE-ISOMERASE. |
| 77 | IPI00008037 | ISOFORM 1 OF LONG-CHAIN-FATTY-ACID--COA LIGASE 5. |
| 78 | IPI00300026 | SULFOTRANSFERASE 1A1. |
| 79 | IPI00005040 | ISOFORM 1 OF MEDIUM-CHAIN SPECIFIC ACYL-COA DEHYDROGENASE, |
| | | MITOCHONDRIAL. |
| 80 | IPI00009532 | CDNA FLJ56034, HIGHLY SIMILAR TO 4-AMINOBUTYRATE |
| | | AMINOTRANSFERASE, MITOCHONDRIAL. |
| 81 | IPI00551024 | BIFUNCTIONAL ATP-DEPENDENT DIHYDROXYACETONE KINASE/FAD-AMP |
| | | LYASE (CYCLIZING). |
| 82 | IPI00014439 | DIHYDROPTERIDINE REDUCTASE. |
| 83 | IPI00219526 | ISOFORM 1 OF PHOSPHOGLUCOMUTASE-1. |
| 84 | IPI00008842 | UPB1 PROTEIN (FRAGMENT). |
| 85 | IPI00007765 | STRESS-70 PROTEIN, MITOCHONDRIAL. |
| 86 | IPI00009268 | CDNA FLJ60317, HIGHLY SIMILAR TO AMINOACYLASE-1. |
| 87 | IPI00759832 | ISOFORM SHORT OF 14-3-3 PROTEIN BETA/ALPHA. |
| 88 | IPI00220642 | 14-3-3 PROTEIN GAMMA. |
| 89 | IPI00216319 | 14-3-3 PROTEIN ETA. |
| 90 | IPI00013890 | ISOFORM 1 OF 14-3-3 PROTEIN SIGMA. |
| 91 | IPI00299402 | PYRUVATE CARBOXYLASE, MITOCHONDRIAL. |
| 92 | IPI00291006 | MALATE DEHYDROGENASE, MITOCHONDRIAL. |
| 93 | IPI00006934 | HYDROXYACID OXIDASE 1. |
| 94 | IPI00002459 | UNCHARACTERIZED PROTEIN. |
| 95 | IPI00006579 | CYTOCHROME C OXIDASE SUBUNIT 4 ISOFORM 1, MITOCHONDRIAL. |
| 96 | IPI00022463 | SEROTRANSFERRIN. |
| 97 | IPI00020984 | CDNA FLJ55574, HIGHLY SIMILAR TO CALNEXIN. |
| 98 | IPI00029715 | ALDEHYDE OXIDASE. |
| 99 | IPI00244391 | XANTHINE DEHYDROGENASE/OXIDASE. |
| 100 | IPI00021405 | ISOFORM A OF PRELAMIN-A/C. |
| 101 | IPI00172593 | ISOFORM 2 OF MUTS PROTEIN HOMOLOG 5. |
| 102 | IPI00218342 | C-1-TETRAHYDROFOLATE SYNTHASE, CYTOPLASMIC. |
| 103 | IPI00550020 | PARATHYMOSIN. |
| 104 | IPI00292709 | PHOSPHOENOLPYRUVATE CARBOXYKINASE, CYTOSOLIC [GTP]. |
| 105 | IPI00002519 | ISOFORM 1 OF SERINE HYDROXYMETHYLTRANSFERASE, CYTOSOLIC. |
| 106 | IPI00021828 | CYSTATIN-B. |
| 107 | IPI01010189 | CDNA FLJ16143 FIS, CLONE BRAMY2038516, HIGHLY SIMILAR TO PROTEIN |
| | | DISULFIDE-ISOMERASE A6. |
| 108 | IPI00186290 | ELONGATION FACTOR 2. |
| 109 | IPI00218831 | ISOFORM 1 OF GLUTATHIONE S-TRANSFERASE MU 1. |
| 110 | IPI00289524 | ALDO-KETO REDUCTASE FAMILY 1 MEMBER C4. |
| 111 | IPI00011229 | CATHEPSIN D. |
| 112 | IPI00021772 | S-ADENOSYLMETHIONINE SYNTHASE ISOFORM TYPE-1. |
| 113 | IPI00000875 | CDNA FLJ56389, HIGHLY SIMILAR TO ELONGATION FACTOR 1-GAMMA. |
| 114 | IPI00028910 | DIHYDROPYRIMIDINASE. |
| 115 | IPI00215901 | ISOFORM 1 OF ADENYLATE KINASE 2, MITOCHONDRIAL. |
| 116 | IPI00013475 | TUBULIN BETA-2A CHAIN. |
| 117 | IPI00003482 | 2,4-DIENOYL-COA REDUCTASE, MITOCHONDRIAL. |
| 118 | IPI00294398 | ISOFORM 1 OF HYDROXYACYL-COENZYME A DEHYDROGENASE, |
| | | MITOCHONDRIAL. |
| 119 | IPI00024933 | ISOFORM 1 OF 60S RIBOSOMAL PROTEIN L12. |
| 120 | IPI00479877 | 4-TRIMETHYLAMINOBUTYRALDEHYDE DEHYDROGENASE. |
| 121 | IPI00783313 | GLYCOGEN PHOSPHORYLASE, LIVER FORM. |
| 122 | IPI00019502 | ISOFORM 1 OF MYOSIN-9. |
| 123 | IPI00908963 | ATP SYNTHASE SUBUNIT ALPHA. |
| 124 | IPI00028031 | CDNA FLJ56425, HIGHLY SIMILAR TO VERY-LONG-CHAIN SPECIFIC ACYL- |
| | | COADEHYDROGENASE, MITOCHONDRIAL. |
| 125 | IPI00022300 | METHYLTRANSFERASE-LIKE PROTEIN 7A. |
| 126 | IPI00219029 | ASPARTATE AMINOTRANSFERASE, CYTOPLASMIC. |
| 127 | IPI00289551 | RETINOL DEHYDROGENASE 16. |
| 128 | IPI00008905 | UDP-GLUCURONOSYLTRANSFERASE 2B15. |
| 129 | IPI00295777 | GLYCEROL-3-PHOSPHATE DEHYDROGENASE [NAD+], CYTOPLASMIC. |
| 130 | IPI00376206 | ISOFORM 2 OF 17-BETA-HYDROXYSTEROID DEHYDROGENASE 13. |
| 131 | IPI00290301 | CYTOCHROME P450 2C8. |
| 132 | IPI00007282 | CYTOCHROME P450 2E1. |
| 133 | IPI00027107 | ELONGATION FACTOR TU, MITOCHONDRIAL PRECURSOR. |
| 134 | IPI00220271 | ALCOHOL DEHYDROGENASE [NADP+]. |
| 135 | IPI00298547 | PROTEIN DJ-1. |
| 136 | IPI00328415 | ISOFORM 1 OF NADH-CYTOCHROME B5 REDUCTASE 3. |
| 137 | IPI00744692 | TRANSALDOLASE. |
| 138 | IPI00032875 | ELECTRON TRANSFER FLAVOPROTEIN-UBIQUINONE OXIDOREDUCTASE, |
| | | MITOCHONDRIAL. |
| 139 | IPI00644771 | ACYL-COENZYME A SYNTHETASE ACSM2A, MITOCHONDRIAL. |
| 140 | IPI00946864 | CDNA FLJ56274, HIGHLY SIMILAR TO TRANSKETOLASE. |
| 141 | IPI00746777 | ALCOHOL DEHYDROGENASE CLASS-3. |
| 142 | IPI00431405 | ISOFORM 2 OF NAD KINASE DOMAIN-CONTAINING PROTEIN 1. |
| 143 | IPI00016513 | RAS-RELATED PROTEIN RAB-10. |
| 144 | IPI00005719 | ISOFORM 1 OF RAS-RELATED PROTEIN RAB-1A. |
| 145 | IPI00292698 | ISOFORM 1 OF ALCOHOL DEHYDROGENASE 6. |
| 146 | IPI00012828 | 3-KETOACYL-COA THIOLASE, PEROXISOMAL. |
| 147 | IPI00293564 | HYDROXYMETHYLGLUTARYL-COA LYASE, MITOCHONDRIAL. |
| 148 | IPI00013808 | ALPHA-ACTININ-4. |
| 149 | IPI00012493 | 40S RIBOSOMAL PROTEIN S20. |
| 150 | IPI00218482 | ISOFORM SHORT OF ES1 PROTEIN HOMOLOG, MITOCHONDRIAL. |
| 151 | IPI00025874 | DOLICHYL-DIPHOSPHOOLIGOSACCHARIDE--PROTEIN GLYCOSYLTRANSFERASE |
| | | SUBUNIT 1 PRECURSOR. |
| 152 | IPI00007219 | CYTOCHROME P450 2C9. |
| 153 | IPI00219525 | 6-PHOSPHOGLUCONATE DEHYDROGENASE, DECARBOXYLATING. |
| 154 | IPI00031131 | ISOFORM 1 OF ADIPOCYTE PLASMA MEMBRANE-ASSOCIATED PROTEIN. |
| 155 | IPI00221091 | 40S RIBOSOMAL PROTEIN S15A. |
| 156 | IPI00005682 | CORTICOSTEROID 11-BETA-DEHYDROGENASE ISOZYME 1. |
| 157 | IPI00028055 | TRANSMEMBRANE EMP24 DOMAIN-CONTAINING PROTEIN 10. |
| 158 | IPI00021842 | APOLIPOPROTEIN E. |
| 159 | IPI00643041 | GTP-BINDING NUCLEAR PROTEIN RAN. |
| 160 | IPI00165360 | 3-MERCAPTOPYRUVATE SULFURTRANSFERASE. |
| 161 | IPI00339319 | ISOFORM 11 OF FIBRONECTIN. |
| 162 | IPI00651653 | PROBABLE ATP-DEPENDENT RNA HELICASE DDX17 ISOFORM 3. |
| 163 | IPI00011253 | 40S RIBOSOMAL PROTEIN S3. |
| 164 | IPI00013917 | 40S RIBOSOMAL PROTEIN S12. |
| 165 | IPI00964764 | CDNA FLJ55072, HIGHLY SIMILAR TO SUCCINATE DEHYDROGENASE |
| | | (UBIQUINONE) FLAVOPROTEIN SUBUNIT, MITOCHONDRIAL. |
| 166 | IPI00009328 | EUKARYOTIC INITIATION FACTOR 4A-III. |
| 167 | IPI00011200 | D-3-PHOSPHOGLYCERATE DEHYDROGENASE. |
| 168 | IPI00032826 | HSC70-INTERACTING PROTEIN. |
| 169 | IPI00026271 | 40S RIBOSOMAL PROTEIN S14. |
| 170 | IPI00383046 | CARBOXYMETHYLENEBUTENOLIDASE HOMOLOG. |
| 171 | IPI00642042 | PUTATIVE UNCHARACTERIZED PROTEIN DKFZP686J1372. |
| 172 | IPI00024067 | ISOFORM 1 OF CLATHRIN HEAVY CHAIN 1. |
| 173 | IPI00337335 | ISOFORM 1 OF MYOSIN-14. |
| 174 | IPI00104341 | CDNA FLJ59619, HIGHLY SIMILAR TO EPOXIDE HYDROLASE 2. |
| 175 | IPI00000690 | ISOFORM 1 OF APOPTOSIS-INDUCING FACTOR 1, MITOCHONDRIAL. |
| 176 | IPI00305360 | AGMATINASE, MITOCHONDRIAL. |
| 177 | IPI00024623 | SHORT/BRANCHED CHAIN SPECIFIC ACYL-COA DEHYDROGENASE, |
| | | MITOCHONDRIAL. |
| 178 | IPI00419237 | ISOFORM 1 OF CYTOSOL AMINOPEPTIDASE. |
| 179 | IPI00216049 | ISOFORM 1 OF HETEROGENEOUS NUCLEAR RIBONUCLEOPROTEIN K. |
| 180 | IPI00303174 | HOMOGENTISATE 1,2-DIOXYGENASE. |
| 181 | IPI00419802 | ISOFORM 1 OF 3-HYDROXYISOBUTYRYL-COA HYDROLASE, MITOCHONDRIAL. |
| 182 | IPI00943181 | UNCHARACTERIZED PROTEIN. |
| 183 | IPI00216951 | ASPARTYL-TRNA SYNTHETASE, CYTOPLASMIC. |
| 184 | IPI00293721 | AFLATOXIN B1 ALDEHYDE REDUCTASE MEMBER 3. |
| 185 | IPI00027701 | SHORT-CHAIN SPECIFIC ACYL-COA DEHYDROGENASE, MITOCHONDRIAL. |
| 186 | IPI00292657 | PROSTAGLANDIN REDUCTASE 1. |
| 187 | IPI00026154 | CDNA FLJ59211, HIGHLY SIMILAR TO GLUCOSIDASE 2 SUBUNIT BETA. |
| 188 | IPI00604620 | NUCLEOLIN. |
| 189 | IPI00030363 | ACETYL-COA ACETYLTRANSFERASE, MITOCHONDRIAL. |
| 190 | IPI00018272 | PYRIDOXINE-5′-PHOSPHATE OXIDASE. |
| 191 | IPI00016610 | POLY(RC)-BINDING PROTEIN 1. |
| 192 | IPI00009375 | ISOFORM 1 OF 3-HYDROXYANTHRANILATE 3,4-DIOXYGENASE. |
| 193 | IPI00024896 | PHENAZINE BIOSYNTHESIS-LIKE DOMAIN-CONTAINING PROTEIN. |
| 194 | IPI00016827 | BILE ACYL-COA SYNTHETASE. |
| 195 | IPI00303954 | CYTOCHROME B5 TYPE B PRECURSOR. |
| 196 | IPI00442121 | ISOFORM 2 OF DELTA-AMINOLEVULINIC ACID DEHYDRATASE. |
| 197 | IPI00549467 | OMEGA-AMIDASE NIT2. |
| 198 | IPI00009368 | SIDEROFLEXIN-1. |
| 199 | IPI00023048 | ISOFORM 1 OF ELONGATION FACTOR 1-DELTA. |
| 200 | IPI00848226 | GUANINE NUCLEOTIDE-BINDING PROTEIN SUBUNIT BETA-2-LIKE 1. |
| 201 | IPI00217975 | LAMIN-B1. |
| 202 | IPI00216592 | ISOFORM Cl OF HETEROGENEOUS NUCLEAR RIBONUCLEOPROTEINS C1/C2. |
| 203 | IPI00215983 | CARBONIC ANHYDRASE 1. |
| 204 | IPI00549725 | PHOSPHOGLYCERATE MUTASE 1. |
| 205 | IPI00009634 | SULFIDE:QUINONE OXIDOREDUCTASE, MITOCHONDRIAL. |
| 206 | IPI00903278 | P37 AUF1. |
| 207 | IPI00413108 | 33 KDA PROTEIN. |
| 208 | IPI00759644 | PEPTIDYL-PROLYL CIS-TRANS ISOMERASE FKBP1A ISOFORM B. |
| 209 | IPI00216691 | PROFILIN-1. |
| 210 | IPI00001734 | PHOSPHOSERINE AMINOTRANSFERASE. |
| 211 | IPI00006443 | ISOFORM 1 OF LAMBDA-CRYSTALLIN HOMOLOG. |
| 212 | IPI00024934 | METHYLMALONYL-COA MUTASE, MITOCHONDRIAL. |
| 213 | IPI00177728 | ISOFORM 1 OF CYTOSOLIC NON-SPECIFIC DIPEPTIDASE. |
| 214 | IPI00178440 | ELONGATION FACTOR 1-BETA. |
| 215 | IPI00376798 | ISOFORM 1 OF 60S RIBOSOMAL PROTEIN L11. |
| 216 | IPI00550363 | TRANSGELIN-2. |
| 217 | IPI00748411 | SERINE HYDROXYMETHYLTRANSFERASE. |
| 218 | IPI00171903 | ISOFORM 1 OF HETEROGENEOUS NUCLEAR RIBONUCLEOPROTEIN M. |
| 219 | IPI00219207 | ISOFORM 3 OF RETICULON-4. |
| 220 | IPI00221222 | ACTIVATED RNA POLYMERASE II TRANSCRIPTIONAL COACTIVATOR P15. |
| 221 | IPI00219153 | 60S RIBOSOMAL PROTEIN L22. |
| 222 | IPI00032258 | COMPLEMENT C4-A. |
| 223 | IPI00465138 | CYTOCHROME P450 3A4. |
| 224 | IPI00217458 | ALANINE AMINOTRANSFERASE 1. |
| 225 | IPI00023542 | TRANSMEMBRANE EMP24 DOMAIN-CONTAINING PROTEIN 9. |
| 226 | IPI00063827 | ISOFORM 1 OF ABHYDROLASE DOMAIN-CONTAINING PROTEIN 14B. |
| 227 | IPI00024145 | ISOFORM 2 OF VOLTAGE-DEPENDENT ANION-SELECTIVE CHANNEL PROTEIN 2. |
| 228 | IPI00909853 | CDNA, FLJ78842, MODERATELY SIMILAR TO D-DOPACHROME |
| | | DECARBOXYLASE. |
| 229 | IPI00015018 | INORGANIC PYROPHOSPHATASE. |
| 230 | IPI00031557 | ISOFORM 1 OF CYSTATHIONINE GAMMA-LYASE. |
| 231 | IPI00215914 | ADP-RIBOSYLATION FACTOR 1. |
| 232 | IPI00026530 | PROTEIN ERGIC-53. |
| 233 | IPI00307246 | ISOFORM 2 OF CYTOCHROME P450 1A2. |
| 234 | IPI00006482 | ISOFORM LONG OF SODIUM/POTASSIUM-TRANSPORTING ATPASE SUBUNIT |
| | | ALPHA-1. |
| 235 | IPI00645078 | UBIQUITIN-LIKE MODIFIER-ACTIVATING ENZYME 1. |
| 236 | IPI00294911 | SUCCINATE DEHYDROGENASE [UBIQUINONE] IRON-SULFUR SUBUNIT, |
| | | MITOCHONDRIAL. |
| 237 | IPI00216136 | ISOFORM C OF KETOHEXOKINASE. |
| 238 | IPI00552715 | T-COMPLEX PROTEIN 1 SUBUNIT GAMMA ISOFORM C. |
| 239 | IPI00383581 | CDNA FLJ61290, HIGHLY SIMILAR TO NEUTRAL ALPHA-GLUCOSIDASE AB. |
| 240 | IPI00025341 | D-BETA-HYDROXYBUTYRATE DEHYDROGENASE, MITOCHONDRIAL. |
| 241 | IPI00843789 | GLYCINE DEHYDROGENASE [DECARBOXYLATING], MITOCHONDRIAL. |
| 242 | IPI00215925 | GLYCINE N-METHYLTRANSFERASE. |
| 243 | IPI00402759 | ISOFORM 1 OF GLYCINE N-ACYLTRANSFERASE. |
| 244 | IPI00411706 | S-FORMYLGLUTATHIONE HYDROLASE. |
| 245 | IPI00329742 | FUMARYLACETOACETATE HYDROLASE DOMAIN-CONTAINING PROTEIN 2A. |
| 246 | IPI00140420 | STAPHYLOCOCCAL NUCLEASE DOMAIN-CONTAINING PROTEIN 1. |
| 247 | IPI00220219 | COATOMER SUBUNIT BETA′. |
| 248 | IPI00215965 | ISOFORM Al-B OF HETEROGENEOUS NUCLEAR RIBONUCLEOPROTEIN Al. |
| 249 | IPI00017579 | PHENYLALANINE-4-HYDROXYLASE. |
| 250 | IPI00026230 | HETEROGENEOUS NUCLEAR RIBONUCLEOPROTEIN H2. |
| 251 | IPI00556579 | MALEYLACETOACETATE ISOMERASE ISOFORM 1. |
| 252 | IPI00219291 | UNCHARACTERIZED PROTEIN. |
| 253 | IPI00293125 | PEROXISOMAL ACYL-COENZYME A OXIDASE 2. |
| 254 | IPI00011603 | 26S PROTEASOME NON-ATPASE REGULATORY SUBUNIT 3. |
| 255 | IPI00179964 | ISOFORM 1 OF POLYPYRIMIDINE TRACT-BINDING PROTEIN 1. |
| 256 | IPI00215918 | ADP-RIBOSYLATION FACTOR 4. |
| 257 | IPI00298971 | VITRONECTIN. |
| 258 | IPI00872799 | ISOFORM 1 OF CYTOCHROME P450 4A11. |
| 259 | IPI00141318 | CYTOSKELETON-ASSOCIATED PROTEIN 4. |
| 260 | IPI00843996 | CDNA FLJ52832, HIGHLY SIMILAR TO SPLICING FACTOR, ARGININE/SERINE- |
| | | RICH 3. |
| 261 | IPI00003990 | ISOFORM 2 OF VALACYCLOVIR HYDROLASE. |
| 262 | IPI00514126 | ISOFORM 1 OF GLYCOGEN DEBRANCHING ENZYME. |
| 263 | IPI00169383 | PHOSPHOGLYCERATE KINASE 1. |
| 264 | IPI00018140 | ISOFORM 1 OF HETEROGENEOUS NUCLEAR RIBONUCLEOPROTEIN Q. |
| 265 | IPI00012074 | ISOFORM 1 OF HETEROGENEOUS NUCLEAR RIBONUCLEOPROTEIN R. |
| 266 | IPI00008524 | ISOFORM 1 OF POLYADENYLATE-BINDING PROTEIN 1. |
| 267 | IPI00479217 | ISOFORM SHORT OF HETEROGENEOUS NUCLEAR RIBONUCLEOPROTEIN U. |
| 268 | IPI00976385 | ENOLASE. |
| 269 | IPI00028635 | DOLICHYL-DIPHOSPHOOLIGOSACCHARIDE--PROTEIN GLYCOSYLTRANSFERASE |
| | | SUBUNIT 2. |
| 270 | IPI00747849 | ISOFORM 1 OF SODIUM/POTASSIUM-TRANSPORTING ATPASE SUBUNIT |
| | | BETA-1. |
| 271 | IPI00043499 | UROCANATE HYDRATASE. |
| 272 | IPI00295363 | ORNITHINE CARBAMOYLTRANSFERASE, MITOCHONDRIAL. |
| 273 | IPI00032103 | ISOFORM 1 OF GLYCINE AMIDINOTRANSFERASE, MITOCHONDRIAL. |
| 274 | IPI00010896 | CHLORIDE INTRACELLULAR CHANNEL PROTEIN 1. |
| 275 | IPI00013296 | 40S RIBOSOMAL PROTEIN S18. |
| 276 | IPI00016339 | RAS-RELATED PROTEIN RAB-5C. |
| 277 | IPI00299000 | PROLIFERATION-ASSOCIATED PROTEIN 2G4. |
| 278 | IPI00218200 | B-CELL RECEPTOR-ASSOCIATED PROTEIN 31. |
| 279 | IPI00296196 | DIMETHYLGLYCINE DEHYDROGENASE, MITOCHONDRIAL. |
| 280 | IPI00844040 | CDNA FLJ59759, HIGHLY SIMILAR TO PROTEIN SET. |
| 281 | IPI00017964 | SMALL NUCLEAR RIBONUCLEOPROTEIN SM D3. |
| 282 | IPI00794561 | CDNA FLJ51998, HIGHLY SIMILAR TO RAS-RELATED PROTEIN RAB-2A. |
| 283 | IPI00296635 | 1,4-ALPHA-GLUCAN-BRANCHING ENZYME. |
| 284 | IPI00000792 | QUINONE OXIDOREDUCTASE. |
| 285 | IPI00291939 | STRUCTURAL MAINTENANCE OF CHROMOSOMES PROTEIN 1A. |
| 286 | IPI00332371 | ISOFORM 1 OF 6-PHOSPHOFRUCTOKINASE, LIVER TYPE. |
| 287 | IPI00219352 | ISOFORM 1 OF CYSTATHIONINE BETA-SYNTHASE. |
| 288 | IPI00295857 | ISOFORM 1 OF COATOMER SUBUNIT ALPHA. |
| 289 | IPI00010740 | ISOFORM LONG OF SPLICING FACTOR, PROLINE- AND GLUTAMINE-RICH. |
| 290 | IPI00473014 | DESTRIN. |
| 291 | IPI00376844 | PUTATIVE UBIQUITIN-CONJUGATING ENZYME E2 N-LIKE. |
| 292 | IPI00027442 | ALANYL-TRNA SYNTHETASE, CYTOPLASMIC. |
| 293 | IPI00299778 | SERUM PARAOXONASE/LACTONASE 3. |
| 294 | IPI00604664 | NADH-UBIQUINONE OXIDOREDUCTASE 75 KDA SUBUNIT, MITOCHONDRIAL |
| | | ISOFORM 5. |
| 295 | IPI00220342 | N(G),N(G)-DIMETHYLARGININE DIMETHYLAMINOHYDROLASE 1. |
| 296 | IPI00002460 | ISOFORM 1 OF ANNEXIN A7. |
| 297 | IPI00019485 | ISOFORM 2 OF ENOYL-COA HYDRATASE DOMAIN-CONTAINING PROTEIN 2, |
| | | MITOCHONDRIAL. |
| 298 | IPI00465256 | GTP:AMP PHOSPHOTRANSFERASE, MITOCHONDRIAL. |
| 299 | IPI00017672 | CDNA FLJ25678 FIS, CLONE TST04067, HIGHLY SIMILAR TO PURINE |
| | | NUCLEOSIDE PHOSPHORYLASE. |
| 300 | IPI00744115 | ISOFORM 1 OF PROPIONYL-COA CARBOXYLASE ALPHA CHAIN, |
| | | MITOCHONDRIAL. |
| 301 | IPI00332828 | COCAINE ESTERASE ISOFORM 1. |
| 302 | IPI00009507 | ISOFORM 1 OF SYNAPTOPHYSIN-LIKE PROTEIN 1. |
| 303 | IPI00029046 | MALECTIN. |
| 304 | IPI00298828 | BETA-2-GLYCOPROTEIN 1. |
| 305 | IPI00021766 | ISOFORM 1 OF RETICULON-4. |
| 306 | IPI00246058 | PROGRAMMED CELL DEATH 6-INTERACTING PROTEIN. |
| 307 | IPI00300567 | ISOFORM 1 OF ENOYL-COA DELTA ISOMERASE 1, MITOCHONDRIAL. |
| 308 | IPI00020956 | HEPATOMA-DERIVED GROWTH FACTOR. |
| 309 | IPI01011543 | CDNA FLJ45429 FIS, CLONE BRHIP3039057, HIGHLY SIMILAR TO PROTEIN |
| | | TRANSPORT PROTEIN SEC23A. |
| 310 | IPI00018465 | T-COMPLEX PROTEIN 1 SUBUNIT ETA. |
| 311 | IPI00412579 | 60S RIBOSOMAL PROTEIN L10A. |
| 312 | IPI00011107 | ISOCITRATE DEHYDROGENASE [NADP], MITOCHONDRIAL. |
| 313 | IPI00914566 | FARNESYL PYROPHOSPHATE SYNTHASE. |
| 314 | IPI00443909 | ISOFORM 1 OF PROTEIN CANOPY HOMOLOG 2. |
| 315 | IPI00022822 | ISOFORM 2 OF COLLAGEN ALPHA-1(XVIII) CHAIN. |
| 316 | IPI00305152 | ISOFORM 3 OF PROTEIN TRANSPORT PROTEIN SEC31A. |
| 317 | IPI00024157 | PEPTIDYL-PROLYL CIS-TRANS ISOMERASE FKBP3. |
| 318 | IPI00900293 | FILAMIN-B ISOFORM 1. |
| 319 | IPI00219678 | EUKARYOTIC TRANSLATION INITIATION FACTOR 2 SUBUNIT 1. |
| 320 | IPI00012912 | CARNITINE O-PALMITOYLTRANSFERASE 2, MITOCHONDRIAL. |
| 321 | IPI00298520 | UNCHARACTERIZED PROTEIN. |
| 322 | IPI00171391 | ISOFORM 1 OF ALDEHYDE DEHYDROGENASE FAMILY 8 MEMBER Al. |
| 323 | IPI00025084 | CALPAIN SMALL SUBUNIT 1. |
| 324 | IPI00009440 | 7-ALPHA-HYDROXYCHOLEST-4-EN-3-ONE 12-ALPHA-HYDROXYLASE. |
| 325 | IPI00217477 | HIGH MOBILITY GROUP PROTEIN B3. |
| 326 | IPI00220834 | X-RAY REPAIR CROSS-COMPLEMENTING PROTEIN 5. |
| 327 | IPI00021890 | ESTRADIOL 17-BETA-DEHYDROGENASE 8. |
| 328 | IPI00029744 | SINGLE-STRANDED DNA-BINDING PROTEIN, MITOCHONDRIAL. |
| 329 | IPI00797126 | UNCHARACTERIZED PROTEIN. |
| 330 | IPI00479786 | ISOFORM 1 OF FAR UPSTREAM ELEMENT-BINDING PROTEIN 2. |
| 331 | IPI00019385 | TRANSLOCON-ASSOCIATED PROTEIN SUBUNIT DELTA. |
| 332 | IPI00220644 | ISOFORM M1 OF PYRUVATE KINASE ISOZYMES M1/M2. |
| 333 | IPI00009960 | ISOFORM 1 OF MITOCHONDRIAL INNER MEMBRANE PROTEIN. |
| 334 | IPI00916847 | 47 KDA PROTEIN. |
| 335 | IPI00215637 | ATP-DEPENDENT RNA HELICASE DDX3X. |
| 336 | IPI00032825 | TRANSMEMBRANE EMP24 DOMAIN-CONTAINING PROTEIN 7. |
| 337 | IPI00644712 | X-RAY REPAIR CROSS-COMPLEMENTING PROTEIN 6. |
| 338 | IPI00030023 | HISTAMINE N-METHYLTRANSFERASE. |
| 339 | IPI00302850 | SMALL NUCLEAR RIBONUCLEOPROTEIN SM D1. |
| 340 | IPI00301021 | ISOFORM 1 OF TRANSLOCON-ASSOCIATED PROTEIN SUBUNIT ALPHA. |
| 341 | IPI00023526 | ISOFORM 1 OF RAS-RELATED PROTEIN RAB-6A. |
| 342 | IPI00783271 | LEUCINE-RICH PPR MOTIF-CONTAINING PROTEIN, MITOCHONDRIAL. |
| 343 | IPI00296913 | ADP-SUGAR PYROPHOSPHATASE. |
| 344 | IPI00305461 | INTER-ALPHA (GLOBULIN) INHIBITOR H2, ISOFORM CRA_A. |
| 345 | IPI00029737 | ISOFORM LONG OF LONG-CHAIN-FATTY-ACID--COA LIGASE 4. |
| 346 | IPI00022228 | VIGILIN. |
| 347 | IPI00302925 | 59 KDA PROTEIN. |
| 348 | IPI00304692 | HETEROGENEOUS NUCLEAR RIBONUCLEOPROTEIN G. |
| 349 | IPI01014975 | UNCHARACTERIZED PROTEIN. |
| 350 | IPI00147874 | SIALIC ACID SYNTHASE. |
| 351 | IPI00011284 | ISOFORM MEMBRANE-BOUND OF CATECHOL O-METHYLTRANSFERASE. |
| 352 | IPI00218015 | ISOFORM 2 OF PROBABLE D-LACTATE DEHYDROGENASE, MITOCHONDRIAL. |
| 353 | IPI00006865 | VESICLE-TRAFFICKING PROTEIN SEC22B. |
| 354 | IPI00927606 | GLUTATHIONE PEROXIDASE 1. |
| 355 | IPI00026302 | 60S RIBOSOMAL PROTEIN L31. |
| 356 | IPI00221354 | ISOFORM SHORT OF RNA-BINDING PROTEIN FUS. |
| 357 | IPI00012007 | ADENOSYLHOMOCYSTEINASE. |
| 358 | IPI00177817 | ISOFORM 2 OF SARCOPLASMIC/ENDOPLASMIC RETICULUM CALCIUM |
| | | ATPASE 2. |
| 359 | IPI00005198 | INTERLEUKIN ENHANCER-BINDING FACTOR 2. |
| 360 | IPI00844578 | ATP-DEPENDENT RNA HELICASE A. |
| 361 | IPI00021805 | MICROSOMAL GLUTATHIONE S-TRANSFERASE 1. |
| 362 | IPI00063234 | UNCHARACTERIZED PROTEIN. |
| 363 | IPI00030781 | ISOFORM ALPHA OF SIGNAL TRANSDUCER AND ACTIVATOR OF |
| | | TRANSCRIPTION 1- ALPHA/BETA. |
| 364 | IPI00007247 | PROPIONYL-COA CARBOXYLASE BETA CHAIN, MITOCHONDRIAL. |
| 365 | IPI00643720 | ISOFORM 1 OF 2-OXOGLUTARATE DEHYDROGENASE-LIKE, MITOCHONDRIAL. |
| 366 | IPI00029631 | ENHANCER OF RUDIMENTARY HOMOLOG. |
| 367 | IPI00100160 | ISOFORM 1 OF CULLIN-ASSOCIATED NEDD8-DISSOCIATED PROTEIN 1. |
| 368 | IPI00018398 | 26S PROTEASE REGULATORY SUBUNIT 6A. |
| 369 | IPI00945507 | SUCCINYL-COA LIGASE [GDP-FORMING] SUBUNIT BETA, MITOCHONDRIAL |
| | | ISOFORM 1 PRECURSOR. |
| 370 | IPI00646917 | CLEAVAGE AND POLYADENYLATION SPECIFICITY FACTOR SUBUNIT 5. |
| 371 | IPI00301936 | CDNA FLJ60076, HIGHLY SIMILAR TO ELAV-LIKE PROTEIN 1. |
| 372 | IPI00017283 | ISOLEUCYL-TRNA SYNTHETASE, MITOCHONDRIAL. |
| 373 | IPI00783982 | COATOMER SUBUNIT GAMMA. |
| 374 | IPI00021435 | 26S PROTEASE REGULATORY SUBUNIT 7. |
| 375 | IPI00024580 | METHYLCROTONOYL-COA CARBOXYLASE SUBUNIT ALPHA, MITOCHONDRIAL. |
| 376 | IPI00008994 | ISOFORM 1 OF PROTEIN NDRG2. |
| 377 | IPI00290279 | ISOFORM LONG OF ADENOSINE KINASE. |
| 378 | IPI00554786 | ISOFORM 5 OF THIOREDOXIN REDUCTASE 1, CYTOPLASMIC. |
| 379 | IPI00009841 | RNA-BINDING PROTEIN EWS ISOFORM 1. |
| 380 | IPI00032220 | ANGIOTENSINOGEN. |
| 381 | IPI00168479 | CDNA FLJ56357, HIGHLY SIMILAR TO HOMO SAPIENS APOLIPOPROTEIN A-I |
| | | BINDING PROTEIN (APOA1BP), MRNA. |
| 382 | IPI00027834 | HETEROGENEOUS NUCLEAR RIBONUCLEOPROTEIN L. |
| 383 | IPI00872762 | SUCCINYL-COA LIGASE [GDP-FORMING] SUBUNIT ALPHA, MITOCHONDRIAL. |
| 384 | IPI00927150 | UNCHARACTERIZED PROTEIN. |
| 385 | IPI00796366 | CDNA FLJ56329, HIGHLY SIMILAR TO MYOSIN LIGHT POLYPEPTIDE 6. |
| 386 | IPI00010130 | GLUTAMINE SYNTHETASE. |
| 387 | IPI00295098 | SIGNAL RECOGNITION PARTICLE RECEPTOR SUBUNIT BETA. |
| 388 | IPI00926977 | 26S PROTEASE REGULATORY SUBUNIT 10B. |
| 389 | IPI00017526 | PROTEIN S100-P. |
| 390 | IPI00008418 | DIABLO HOMOLOG, MITOCHONDRIAL PRECURSOR. |
| 391 | IPI00009950 | VESICULAR INTEGRAL-MEMBRANE PROTEIN VIP36. |
| 392 | IPI00030702 | ISOFORM 1 OF ISOCITRATE DEHYDROGENASE [NAD] SUBUNIT ALPHA, |
| | | MITOCHONDRIAL. |
| 393 | IPI00009032 | LUPUS LA PROTEIN. |
| 394 | IPI00003881 | HETEROGENEOUS NUCLEAR RIBONUCLEOPROTEIN F. |
| 395 | IPI00026167 | NHP2-LIKE PROTEIN 1. |
| 396 | IPI00024466 | ISOFORM 1 OF UDP-GLUCOSE:GLYCOPROTEIN GLUCOSYLTRANSFERASE 1. |
| 397 | IPI00027285 | ISOFORM SM-B′ OF SMALL NUCLEAR RIBONUCLEOPROTEIN-ASSOCIATED |
| | | PROTEINS B AND B′. |
| 398 | IPI00219330 | ISOFORM 5 OF INTERLEUKIN ENHANCER-BINDING FACTOR 3. |
| 399 | IPI00030962 | ISOFORM 5 OF UBIQUITIN-CONJUGATING ENZYME E2 VARIANT 1. |
| 400 | IPI00007940 | ERLIN-1. |
| 401 | IPI00030131 | ISOFORM BETA OF LAMINA-ASSOCIATED POLYPEPTIDE 2, ISOFORMS |
| | | BETA/GAMMA. |
| 402 | IPI00001639 | IMPORTIN SUBUNIT BETA-1. |
| 403 | IPI00002966 | HEAT SHOCK 70 KDA PROTEIN 4. |
| 404 | IPI00000873 | VALYL-TRNA SYNTHETASE. |
| 405 | IPI00449049 | POLY [ADP-RIBOSE] POLYMERASE 1. |
| 406 | IPI00419880 | 40S RIBOSOMAL PROTEIN S3A. |
| 407 | IPI00289499 | BIFUNCTIONAL PURINE BIOSYNTHESIS PROTEIN PURH. |
| 408 | IPI00028520 | ISOFORM 1 OF NADH DEHYDROGENASE [UBIQUINONE] FLAVOPROTEIN 1, |
| | | MITOCHONDRIAL. |
| 409 | IPI00550689 | TRNA-SPLICING LIGASE RTCB HOMO LOG. |
| 410 | IPI00396370 | ISOFORM 1 OF EUKARYOTIC TRANSLATION INITIATION FACTOR 3 SUBUNIT B. |
| 411 | IPI00015602 | MITOCHONDRIAL IMPORT RECEPTOR SUBUNIT TOM70. |
| 412 | IPI00413778 | FKBP1A PROTEIN. |
| 413 | IPI00219129 | RIBOSYLDIHYDRONICOTINAMIDE DEHYDROGENASE [QUINONE]. |
| 414 | IPI00984829 | PROTEIN. |
| 415 | IPI00170796 | ISOFORM 1 OF VACUOLAR PROTEIN SORTING-ASSOCIATED PROTEIN 29. |
| 416 | IPI00020672 | ISOFORM 1 OF DIPEPTIDYL PEPTIDASE 3. |
| 417 | IPI00019591 | CDNA FLJ55673, HIGHLY SIMILAR TO COMPLEMENT FACTOR B. |
| 418 | IPI00017551 | ISOFORM 1 OF REGUCALCIN. |
| 419 | IPI00019407 | STEROL-4-ALPHA-CARBOXYLATE 3-DEHYDROGENASE, DECARBOXYLATING. |
| 420 | IPI00217952 | ISOFORM 1 OF GLUCOSAMINE--FRUCTOSE-6-PHOSPHATE |
| | | AMINOTRANSFERASE [ISOMERIZING] 1. |
| 421 | IPI00030182 | GUANIDINOACETATE N-METHYLTRANSFERASE. |
| 422 | IPI00550644 | TETRATRICOPEPTIDE REPEAT PROTEIN 38. |
| 423 | IPI00328748 | CDNA FLJ77177, HIGHLY SIMILAR TO HOMO SAPIENS ARGININE-RICH, |
| | | MUTATED IN EARLY STAGE TUMORS (ARMET), MRNA. |
| 424 | IPI00291328 | NADH DEHYDROGENASE [UBIQUINONE] FLAVOPROTEIN 2, MITOCHONDRIAL. |
| 425 | IPI00305692 | THIOREDOXIN-LIKE PROTEIN 1. |
| 426 | IPI00004373 | MANNOSE-BINDING PROTEIN C. |
| 427 | IPI00006114 | PIGMENT EPITHELIUM-DERIVED FACTOR. |
| 428 | IPI00292858 | THYMIDINE PHOSPHORYLASE. |
| 429 | IPI00056369 | ISOFORM 1 OF GLYCINE N-ACYLTRANSFERASE-LIKE PROTEIN 1. |
| 430 | IPI00219913 | UBIQUITIN CARBOXYL-TERMINAL HYDROLASE 14. |
| 431 | IPI00297635 | ISOFORM 1 OF ACYL-COENZYME A SYNTHETASE ACSM3, MITOCHONDRIAL. |
| 432 | IPI00005614 | ISOFORM LONG OF SPECTRIN BETA CHAIN, BRAIN 1. |
| 433 | IPI00003519 | 116 KDA U5 SMALL NUCLEAR RIBONUCLEOPROTEIN COMPONENT. |
| 434 | IPI00001466 | ECHINODERM MICROTUBULE-ASSOCIATED PROTEIN-LIKE 4. |
| 435 | IPI00155601 | MACRO DOMAIN-CONTAINING PROTEIN 1. |
| 436 | IPI00105598 | PROTEASOME 26S NON-ATPASE SUBUNIT 11 VARIANT (FRAGMENT). |
| 437 | IPI00645307 | ISOFORM 1 OF ISOPENTENYL-DIPHOSPHATE DELTA-ISOMERASE 1. |
| 438 | IPI00012645 | ISOFORM 1 OF SPECTRIN BETA CHAIN, BRAIN 2. |
| 439 | IPI00009235 | TRANSLOCON-ASSOCIATED PROTEIN SUBUNIT GAMMA. |
| 440 | IPI00182469 | ISOFORM 1AB OF CATENIN DELTA-1. |
| 441 | IPI00412498 | UNCHARACTERIZED PROTEIN. |
| 442 | IPI00910745 | CDNA FLJ58224, HIGHLY SIMILAR TO CALPAIN-2 CATALYTIC SUBUNIT. |
| 443 | IPI00004860 | ISOFORM COMPLEXED OF ARGINYL-TRNA SYNTHETASE, CYTOPLASMIC. |
| 444 | IPI00029012 | EUKARYOTIC TRANSLATION INITIATION FACTOR 3 SUBUNIT A. |
| 445 | IPI00027341 | MACROPHAGE-CAPPING PROTEIN. |
| 446 | IPI00022432 | TRANSTHYRETIN. |
| 447 | IPI00376344 | ISOFORM 1 OF MYOSIN-IB. |
| 448 | IPI00219005 | PEPTIDYL-PROLYL CIS-TRANS ISOMERASE FKBP4. |
| 449 | IPI00293655 | ATP-DEPENDENT RNA HELICASE DDX1. |
| 450 | IPI00003933 | ISOFORM 1 OF HYDROXYACYLGLUTATHIONE HYDROLASE, MITOCHONDRIAL. |
| 451 | IPI00217420 | ISOFORM 2 OF HYDROXYACID-OXOACID TRANSHYDROGENASE, |
| | | MITOCHONDRIAL. |
| 452 | IPI00399318 | COATOMER SUBUNIT EPSILON ISOFORM B. |
| 453 | IPI00101652 | SELENOCYSTEINE LYASE. |
| 454 | IPI00925046 | GLUTAMINYL-TRNA SYNTHETASE. |
| 455 | IPI00220327 | KERATIN, TYPE II CYTOSKELETAL 1. |
| 456 | IPI00784029 | ISOFORM 1 OF OXIDOREDUCTASE HTATIP2. |
| 457 | IPI00296337 | ISOFORM 1 OF DNA-DEPENDENT PROTEIN KINASE CATALYTIC SUBUNIT. |
| 458 | IPI00029629 | E3 UBIQUITIN/ISG15 LIGASE TRIM25. |
| 459 | IPI00012268 | 26S PROTEASOME NON-ATPASE REGULATORY SUBUNIT 2. |
| 460 | IPI00301503 | ISOFORM 1 OF TRANSFORMER-2 PROTEIN HOMOLOG BETA. |
| 461 | IPI00023860 | NUCLEOSOME ASSEMBLY PROTEIN 1-LIKE 1. |
| 462 | IPI00008380 | SERINE/THREONINE-PROTEIN PHOSPHATASE 2A CATALYTIC SUBUNIT ALPHA |
| | | ISOFORM. |
| 463 | IPI00014808 | PLATELET-ACTIVATING FACTOR ACETYLHYDROLASE IB SUBUNIT GAMMA. |
| 464 | IPI00217253 | GTP CYCLOHYDROLASE 1 FEEDBACK REGULATORY PROTEIN. |
| 465 | IPI00305698 | VITAMIN K-DEPENDENT GAMMA-CARBOXYLASE. |
| 466 | IPI00219861 | ISOFORM 1 OF LOW MOLECULAR WEIGHT PHOSPHOTYROSINE PROTEIN |
| | | PHOSPHATASE. |
| 467 | IPI00017297 | MATRIN-3. |
| 468 | IPI00033022 | ISOFORM 1 OF DYNAMIN-2. |
| 469 | IPI00029739 | ISOFORM 1 OF COMPLEMENT FACTOR H. |
| 470 | IPI00001757 | ISOFORM 1 OF RNA-BINDING PROTEIN 8A. |
| 471 | IPI00216125 | ISOFORM 2 OF SIGNAL RECOGNITION PARTICLE 9 KDA PROTEIN. |
| 472 | IPI00021808 | HISTIDYL-TRNA SYNTHETASE, CYTOPLASMIC. |
| 473 | IPI00219512 | ISOFORM 2 OF UBIQUITIN CARBOXYL-TERMINAL HYDROLASE ISOZYME L5. |
| 474 | IPI00215948 | ISOFORM 1 OF CATENIN ALPHA-1. |
| 475 | IPI00479306 | ISOFORM 1 OF PROTEASOME SUBUNIT BETA TYPE-5. |
| 476 | IPI00018931 | VACUOLAR PROTEIN SORTING-ASSOCIATED PROTEIN 35. |
| 477 | IPI00021290 | ATP-CITRATE SYNTHASE. |
| 478 | IPI00385901 | ISOFORM 2 OF UBIQUINONE BIOSYNTHESIS PROTEIN COQ9, |
| | | MITOCHONDRIAL. |
| 479 | IPI00413674 | ISOFORM 1 OF PHYTANOYL-COA DIOXYGENASE DOMAIN-CONTAINING |
| | | PROTEIN 1. |
| 480 | IPI00293434 | SIGNAL RECOGNITION PARTICLE 14 KDA PROTEIN. |
| 481 | IPI00034308 | SARCOSINE DEHYDROGENASE, MITOCHONDRIAL. |
| 482 | IPI00295940 | CDNA FLJ55508, HIGHLY SIMILAR TO SAD1/UNC-84-LIKE PROTEIN 2. |
| 483 | IPI00383879 | ARYLACETAMIDE DEACETYLASE. |
| 484 | IPI00975644 | UNCHARACTERIZED PROTEIN. |
| 485 | IPI00297982 | EUKARYOTIC TRANSLATION INITIATION FACTOR 2 SUBUNIT 3. |
| 486 | IPI00008485 | CYTOPLASMIC ACONITATE HYDRATASE. |
| 487 | IPI00304596 | NON-POU DOMAIN-CONTAINING OCTAMER-BINDING PROTEIN. |
| 488 | IPI00005160 | ACTIN-RELATED PROTEIN 2/3 COMPLEX SUBUNIT 1B. |
| 489 | IPI00873506 | GUANINE AMINOHYDROLASE. |
| 490 | IPI00396435 | PUTATIVE PRE-MRNA-SPLICING FACTOR ATP-DEPENDENT RNA HELICASE |
| | | DHX15. |
| 491 | IPI00001699 | ISOFORM 1 OF APOPTOSIS-ASSOCIATED SPECK-LIKE PROTEIN CONTAINING A |
| | | CARD. |
| 492 | IPI00446769 | ISOFORM 2 OF 3-HYDROXYBUTYRATE DEHYDROGENASE TYPE 2. |
| 493 | IPI00745906 | SULFITE OXIDASE, MITOCHONDRIAL. |
| 494 | IPI00017451 | SPLICING FACTOR 3A SUBUNIT 1. |
| 495 | IPI00328753 | ISOFORM 1 OF KINECTIN. |
| 496 | IPI00942092 | ISOFORM 1 OF ADENYLOSUCCINATE LYASE. |
| 497 | IPI00333985 | ISOFORM 2 OF NODAL MODULATOR 2. |
| 498 | IPI00216298 | THIOREDOXIN. |
| 499 | IPI00295772 | LANOSTEROL 14-ALPHA DEMETHYLASE ISOFORM 1. |
| 500 | IPI00022143 | ISOFORM 1 OF EXTENDED SYNAPTOTAGMIN-1. |
| 501 | IPI00025815 | ISOFORM 2 OF TAR DNA-BINDING PROTEIN 43. |
| 502 | IPI00019927 | 26S PROTEASOME NON-ATPASE REGULATORY SUBUNIT 7. |
| 503 | IPI00013452 | BIFUNCTIONAL AMINOACYL-TRNA SYNTHETASE. |
| 504 | IPI00008240 | METHIONYL-TRNA SYNTHETASE, CYTOPLASMIC. |
| 505 | IPI00024284 | BASEMENT MEMBRANE-SPECIFIC HEPARAN SULFATE PROTEOGLYCAN CORE |
| | | PROTEIN. |
| 506 | IPI00220267 | ARGININOSUCCINATE LYASE. |
| 507 | IPI00395627 | ISOFORM 1 OF CALCYCLIN-BINDING PROTEIN. |
| 508 | IPI00550523 | ATLASTIN-3. |
| 509 | IPI00012535 | DNAJ HOMOLOG SUBFAMILY A MEMBER 1. |
| 510 | IPI00300074 | PHENYLALANYL-TRNA SYNTHETASE BETA CHAIN. |
| 511 | IPI00456969 | CYTOPLASMIC DYNEIN 1 HEAVY CHAIN 1 |
| 512 | IPI00022429 | ALPHA-1-ACID GLYCOPROTEIN 1. |
| 513 | IPI00009367 | SERINE--PYRUVATE AMINOTRANSFERASE. |
| 514 | IPI00220710 | ISOFORM 1 OF ACYL-COENZYME A THIOESTERASE 9, MITOCHONDRIAL. |
| 515 | IPI00433508 | CYTOCHROME P450 2D6 ISOFORM 2. |
| 516 | IPI00026105 | ISOFORM SCPX OF NON-SPECIFIC LIPID-TRANSFER PROTEIN. |
| 517 | IPI00477957 | ISOFORM 1 OF CITRATE LYASE SUBUNIT BETA-LIKE PROTEIN, |
| | | MITOCHONDRIAL. |
| 518 | IPI00293126 | TUBULIN-FOLDING COFACTOR B. |
| 519 | IPI00297477 | U2 SMALL NUCLEAR RIBONUCLEOPROTEIN A′. |
| 520 | IPI00024661 | PROTEIN TRANSPORT PROTEIN SEC24C. |
| 521 | IPI00297579 | CHROMOBOX PROTEIN HOMOLOG 3. |
| 522 | IPI00554742 | ISOFORM 2 OF APOPTOSIS INHIBITOR 5. |
| 523 | IPI00514301 | ISOFORM 1 OF PERIPHERAL PLASMA MEMBRANE PROTEIN CASK. |
| 524 | IPI00746165 | ISOFORM 1 OF WD REPEAT-CONTAINING PROTEIN 1 |
| 525 | IPI00220373 | INSULIN-DEGRADING ENZYME. |
| 526 | IPI00011250 | UBIQUITIN CARBOXYL-TERMINAL HYDROLASE ISOZYME L3. |
| 527 | IPI00011274 | ISOFORM 1 OF HETEROGENEOUS NUCLEAR RIBONUCLEOPROTEIN D-LIKE. |
| 528 | IPI00009253 | ALPHA-SOLUBLE NSF ATTACHMENT PROTEIN. |
| 529 | IPI00429191 | EUKARYOTIC PEPTIDE CHAIN RELEASE FACTOR SUBUNIT 1. |
| 530 | IPI00186704 | N-ACETYLGALACTOSAMINE KINASE ISOFORM 2. |
| 531 | IPI00917623 | UNCHARACTERIZED PROTEIN. |
| 532 | IPI00043911 | PROBABLE IMIDAZOLONEPROPIONASE. |
| 533 | IPI00000816 | ISOFORM 1 OF 14-3-3 PROTEIN EPSILON. |
| 534 | IPI00011604 | GLYCINE CLEAVAGE SYSTEM H PROTEIN, MITOCHONDRIAL. |
| 535 | IPI00292020 | SPERMIDINE SYNTHASE. |
| 536 | IPI00061525 | GLUCOSAMINE 6-PHOSPHATE N-ACETYLTRANSFERASE. |
| 537 | IPI00880007 | MICROTUBULE-ASSOCIATED PROTEIN. |
| 538 | IPI00032311 | LIPOPOLYSACCHARIDE-BINDING PROTEIN. |
| 539 | IPI00022201 | L-SERINE DEHYDRATASE/L-THREONINE DEAMINASE. |
| 540 | IPI00300371 | ISOFORM 1 OF SPLICING FACTOR 3B SUBUNIT 3. |
| 541 | IPI00013933 | ISOFORM DPI OF DESMOPLAKIN. |
| 542 | IPI00221224 | AMINOPEPTIDASE N. |
| 543 | IPI00924544 | 35 KDA PROTEIN. |
| 544 | IPI00299149 | SMALL UBIQUITIN-RELATED MODIFIER 2. |
| 545 | IPI00219111 | TRANSLOCATING CHAIN-ASSOCIATED MEMBRANE PROTEIN 1. |
| 546 | IPI00221178 | ISOFORM 2 OF TUMOR PROTEIN D54. |
| 547 | IPI00009923 | ISOFORM 1 OF PROLYL 4-HYDROXYLASE SUBUNIT ALPHA-1. |
| 548 | IPI00328319 | ISOFORM 1 OF HISTONE-BINDING PROTEIN RBBP4. |
| 549 | IPI00006167 | PROTEIN PHOSPHATASE 1G. |
| 550 | IPI00106509 | ISOFORM 4 OF HETEROGENEOUS NUCLEAR RIBONUCLEOPROTEIN A/B. |
| 551 | IPI00024976 | MITOCHONDRIAL IMPORT RECEPTOR SUBUNIT TOM22 HOMOLOG. |
| 552 | IPI00291643 | SPRY DOMAIN-CONTAINING PROTEIN 4. |
| 553 | IPI00374657 | ISOFORM 2 OF VESICLE-ASSOCIATED MEMBRANE PROTEIN-ASSOCIATED |
| | | PROTEIN A. |
| 554 | IPI00011916 | AMINOACYL TRNA SYNTHASE COMPLEX-INTERACTING MULTIFUNCTIONAL |
| | | PROTEIN 2. |
| 555 | IPI00294578 | ISOFORM 1 OF PROTEIN-GLUTAMINE GAMMA-GLUTAMYLTRANSFERASE 2. |
| 556 | IPI00000015 | SERINE/ARGININE-RICH SPLICING FACTOR 4. |
| 557 | IPI00306382 | ISOFORM 1 OF SECRETORY CARRIER-ASSOCIATED MEMBRANE PROTEIN 3. |
| 558 | IPI00179057 | ISOFORM 2 OF CULLIN-4B. |
| 559 | IPI00022830 | ISOFORM 2 OF NSFL1 COFACTOR P47. |
| 560 | IPI00008454 | DNAJ HOMOLOG SUBFAMILY B MEMBER 11. |
| 561 | IPI00064328 | PROTEIN ARGININE N-METHYLTRANSFERASE 5 ISOFORM B. |
| 562 | IPI00032460 | U6 SNRNA-ASSOCIATED SM-LIKE PROTEIN LSM2. |
| 563 | IPI00013495 | ISOFORM 2 OF ATP-BINDING CASSETTE SUB-FAMILY F MEMBER 1. |
| 564 | IPI00165230 | ISOFORM 1 OF DAZ-ASSOCIATED PROTEIN 1. |
| 565 | IPI00294879 | RAN GTPASE-ACTIVATING PROTEIN 1. |
| 566 | IPI00479997 | ISOFORM 1 OF STATHMIN. |
| 567 | IPI00163230 | COP9 SIGNALOSOME COMPLEX SUBUNIT 6. |
| 568 | IPI00013174 | ISOFORM 1 OF RNA-BINDING PROTEIN 14. |
| 569 | IPI00374563 | AGRIN. |
| 570 | IPI00306960 | ASPARAGINYL-TRNA SYNTHETASE, CYTOPLASMIC. |
| 571 | IPI00021187 | ISOFORM 1 OF RUVB-LIKE 1. |
| 572 | IPI00009943 | TUMOR PROTEIN, TRANSLATIONALLY-CONTROLLED 1. |
| 573 | IPI00438229 | ISOFORM 1 OF TRANSCRIPTION INTERMEDIARY FACTOR 1-BETA. |
| 574 | IPI00004457 | MEMBRANE PRIMARY AMINE OXIDASE. |
| 575 | IPI00022462 | TRANSFERRIN RECEPTOR PROTEIN 1. |
| 576 | IPI00009704 | PROBABLE PROLINE DEHYDROGENASE 2. |
| 577 | IPI00024417 | HUNTINGTIN-INTERACTING PROTEIN 1-RELATED PROTEIN. |
| 578 | IPI00414860 | 60S RIBOSOMAL PROTEIN L37A. |
| 579 | IPI00029601 | SRC SUBSTRATE CORTACTIN. |
| 580 | IPI00015736 | ISOFORM 1 OF UBIQUITIN-LIKE MODIFIER-ACTIVATING ENZYME 5. |
| 581 | IPI00006721 | ISOFORM 1 OF DYNAMIN-LIKE 120 KDA PROTEIN, MITOCHONDRIAL. |
| 582 | IPI00295851 | COATOMER SUBUNIT BETA. |
| 583 | IPI00217236 | TUBULIN-SPECIFIC CHAPERONE A. |
| 584 | IPI00045051 | TRANSCRIPTIONAL ACTIVATOR PROTEIN PUR-BETA. |
| 585 | IPI00006207 | ISOFORM 2 OF LEUCINE-RICH REPEAT FLIGHTLESS-INTERACTING PROTEIN 1. |
| 586 | IPI00021695 | ISOFORM D OF PLASMA MEMBRANE CALCIUM-TRANSPORTING ATPASE 1. |
| 587 | IPI00418497 | ISOFORM 2 OF MITOCHONDRIAL IMPORT INNER MEMBRANE TRANSLOCASE |
| | | SUBUNIT TIM50. |
| 588 | IPI00016287 | THREONINE SYNTHASE-LIKE 1. |
| 589 | IPI00294536 | CDNA FLJ51909, HIGHLY SIMILAR TO SERINE-THREONINE KINASE RECEPTOR- |
| | | ASSOCIATEDPROTEIN. |
| 590 | IPI00013070 | ISOFORM 1 OF HETEROGENEOUS NUCLEAR RIBONUCLEOPROTEIN U-LIKE |
| | | PROTEIN 1. |
| 591 | IPI00619898 | NQO1 PROTEIN (FRAGMENT). |
| 592 | IPI00008867 | GLYCOGEN [STARCH] SYNTHASE, LIVER. |
| 593 | IPI00021466 | ISOFORM 1 OF POLYADENYLATE-BINDING PROTEIN-INTERACTING PROTEIN 1. |
| 594 | IPI00000581 | CDNA FLJ56307, HIGHLY SIMILAR TO UBIQUITIN THIOESTERASE PROTEIN |
| | | OTUB1. |
| 595 | IPI00018120 | 28S RIBOSOMAL PROTEIN S29, MITOCHONDRIAL. |
| 596 | IPI00554590 | ISOFORM 1 OF RAB3 GTPASE-ACTIVATING PROTEIN NON-CATALYTIC |
| | | SUBUNIT. |
| 597 | IPI00220528 | SMALL NUCLEAR RIBONUCLEOPROTEIN F. |
| 598 | IPI00978402 | UNCHARACTERIZED PROTEIN. |
| 599 | IPI00025057 | ISOFORM 2 OF DOUBLE-STRANDED RNA-SPECIFIC ADENOSINE DEAMINASE. |
| 600 | IPI00910005 | CDNA FLJ59832, MODERATELY SIMILAR TO PROSTAGLANDIN E SYNTHASE 3. |
| 601 | IPI00069750 | ISOFORM 1 OF POLY(U)-BINDING-SPLICING FACTOR PUF60. |
| 602 | IPI00004928 | ISOFORM 1 OF EGL NINE HOMOLOG 1. |
| 603 | IPI00013925 | GALACTOSE-1-PHOSPHATE URIDYLYLTRANSFERASE. |
| 604 | IPI00000663 | ISOFORM MITOCHONDRIAL OF MALONYL-COA DECARBOXYLASE, |
| | | MITOCHONDRIAL. |
| 605 | IPI00152441 | ISOFORM 1 OF MINOR HISTOCOMPATIBILITY ANTIGEN H13. |
| 606 | IPI00008982 | ISOFORM LONG OF DELTA-1-PYRROLINE-5-CARBOXYLATE SYNTHASE. |
| 607 | IPI00163505 | ISOFORM 1 OF RNA-BINDING PROTEIN 39. |
| 608 | IPI00009659 | REGULATION OF NUCLEAR PRE-MRNA DOMAIN-CONTAINING PROTEIN 1B. |
| 609 | IPI00103994 | LEUCYL-TRNA SYNTHETASE, CYTOPLASMIC. |
| 610 | IPI00220993 | ISOFORM CNPI OF 2′,3′-CYCLIC-NUCLEOTIDE 3′-PHOSPHODIESTERASE. |
| 611 | IPI00219156 | 60S RIBOSOMAL PROTEIN L30. |
| 612 | IPI00004968 | PRE-MRNA-PROCESSING FACTOR 19. |
| 613 | IPI00032140 | SERPIN H1. |
| 614 | IPI00293088 | LYSOSOMAL ALPHA-GLUCOSIDASE. |
| 615 | IPI00843975 | EZRIN. |
| 616 | IPI00290452 | TRANSMEMBRANE BAX INHIBITOR MOTIF-CONTAINING PROTEIN 1. |
| 617 | IPI00745502 | 26S PROTEASE REGULATORY SUBUNIT 8 ISOFORM 2. |
| 618 | IPI00298860 | CDNA FLJ78440, HIGHLY SIMILAR TO HUMAN LACTOFERRIN. |
| 619 | IPI00032003 | EMERIN. |
| 620 | IPI00024825 | ISOFORM A OF PROTEOGLYCAN 4. |
| 621 | IPI00006181 | EUKARYOTIC TRANSLATION INITIATION FACTOR 3 SUBUNIT D. |
| 622 | IPI00025831 | CYTOCHROME P450 3A5. |
| 623 | IPI00019907 | GLYPICAN-3. |
| 624 | IPI00166092 | N-ACETYLGLUTAMATE SYNTHASE, MITOCHONDRIAL. |
| 625 | IPI00163849 | CDNA FLJ60624, HIGHLY SIMILAR TO EPIDERMAL GROWTH FACTOR |
| | | RECEPTOR SUBSTRATE 15-LIKE 1. |
| 626 | IPI00298281 | LAMININ SUBUNIT GAMMA-1. |
| 627 | IPI00018350 | DNA REPLICATION LICENSING FACTOR MCM5. |
| 628 | IPI00025100 | 2-OXOISOVALERATE DEHYDROGENASE SUBUNIT ALPHA, MITOCHONDRIAL. |
| 629 | IPI00419903 | ISOFORM 1 OF PUTATIVE L-ASPARTATE DEHYDROGENASE. |
| 630 | IPI00183208 | ISOFORM 1 OF F-BOX ONLY PROTEIN 22. |
| 631 | IPI00014149 | TETRATRICOPEPTIDE REPEAT PROTEIN 35. |
| 632 | IPI00329633 | THREONYL-TRNA SYNTHETASE, CYTOPLASMIC. |
| 633 | IPI00644231 | ISOFORM 1 OF CYTOPLASMIC FMR1-INTERACTING PROTEIN 1. |
| 634 | IPI00002147 | CHITINASE-3-LIKE PROTEIN 1. |
| 635 | IPI00216008 | ISOFORM LONG OF GLUCOSE-6-PHOSPHATE 1-DEHYDROGENASE. |
| 636 | IPI00003309 | DNA-DIRECTED RNA POLYMERASES I, II, AND III SUBUNIT RPABC3. |
| 637 | IPI00178798 | ISOFORM 2 OF PROTEIN TRANSPORT PROTEIN SEC24A. |
| 638 | IPI00176469 | ISOFORM 1 OF CHAPERONE ACTIVITY OF BC1 COMPLEX-LIKE, |
| | | MITOCHONDRIAL. |
| 639 | IPI00019848 | ISOFORM 1 OF HOST CELL FACTOR 1. |
| 640 | IPI00005794 | UNCHARACTERIZED PROTEIN. |
| 641 | IPI00008613 | ISOFORM 1 OF INTERFERON-INDUCED 35 KDA PROTEIN. |
| 642 | IPI00012970 | ISOFORM 1 OF SERINE/THREONINE-PROTEIN PHOSPHATASE 6 CATALYTIC |
| | | SUBUNIT. |
| 643 | IPI00023234 | SUMO-ACTIVATING ENZYME SUBUNIT 2. |
| 644 | IPI00789155 | CALUMENIN ISOFORM C PRECUROSR. |
| 645 | IPI00298961 | EXPORTIN-1. |
| 646 | IPI00032881 | 28S RIBOSOMAL PROTEIN S23, MITOCHONDRIAL. |
| 647 | IPI00013214 | CDNA FLJ55599, HIGHLY SIMILAR TO DNA REPLICATION LICENSING FACTOR |
| | | MCM3. |
| 648 | IPI00791534 | SOLUTE CARRIER FAMILY 4, ANION EXCHANGER, MEMBER 1. |
| 649 | IPI00006196 | ISOFORM 2 OF NUCLEAR MITOTIC APPARATUS PROTEIN 1. |
| 650 | IPI00025039 | RRNA 2′-O-METHYLTRANSFERASE FIBRILLARIN. |
| 651 | IPI00007166 | IMMEDIATE EARLY RESPONSE 3-INTERACTING PROTEIN 1. |
| 652 | IPI00218925 | ISOFORM 1 OF PEROXISOMAL MEMBRANE PROTEIN 4. |
| 653 | IPI00215687 | ISOFORM 3 OF GLUTAMINASE KIDNEY ISOFORM, MITOCHONDRIAL. |
| 654 | IPI00018632 | CDNA FLJ12528 FIS, CLONE NT2RM4000155, MODERATELY SIMILAR TO |
| | | THREONYL- TRNA SYNTHETASE, CYTOPLASMIC. |
| 655 | IPI00003870 | PUTATIVE ATP-DEPENDENT CLP PROTEASE PROTEOLYTIC SUBUNIT, |
| | | MITOCHONDRIAL. |
| 656 | IPI00646954 | 89 KDA PROTEIN. |
| 657 | IPI00292695 | LONG-CHAIN SPECIFIC ACYL-COA DEHYDROGENASE, MITOCHONDRIAL. |
| 658 | IPI00016568 | ADENYLATE KINASE ISOENZYME 4, MITOCHONDRIAL. |
| 659 | IPI00021570 | ISOFORM 1 OF ENDOTHELIAL DIFFERENTIATION-RELATED FACTOR 1. |
| 660 | IPI01016029 | ISOFORM 2 OF MITOCHONDRIAL PEPTIDE METHIONINE SULFOXIDE |
| | | REDUCTASE. |
| 661 | IPI00022095 | ISOFORM RF1/RF2 OF RETROTRANSPOSON-DERIVED PROTEIN PEG10. |
| 662 | IPI00022078 | PROTEIN NDRG1. |
| 663 | IPI00013774 | HISTONE DEACETYLASE 1. |
| 664 | IPI00013079 | EMILIN-1. |
| 665 | IPI00006722 | ISOFORM 1 OF PEROXISOMAL MEMBRANE PROTEIN PEX16. |
| 666 | IPI00168262 | PROCOLLAGEN GALACTOSYLTRANSFERASE 1. |
| 667 | IPI00031556 | ISOFORM 1 OF SPLICING FACTOR U2AF 65 KDA SUBUNIT. |
| 668 | IPI00103525 | ISOFORM 1 OF PARASPECKLE COMPONENT 1. |
| 669 | IPI00478961 | ISOFORM 1 OF FGGY CARBOHYDRATE KINASE DOMAIN-CONTAINING |
| | | PROTEIN. |
| 670 | IPI00028908 | ISOFORM 1 OF NIDOGEN-2. |
| 671 | IPI00982620 | CDNA FLJ61765, HIGHLY SIMILAR TO 4-TRIMETHYLAMINOBUTYRALDEHYDE |
| | | DEHYDROGENASE. |
| 672 | IPI00472604 | UNCHARACTERIZED PROTEIN. |
| 673 | IPI00414612 | ISOFORM 1 OF PUTATIVE HEXOKINASE HKDC1. |
| 674 | IPI00412713 | SORTING AND ASSEMBLY MACHINERY COMPONENT 50 HOMOLOG. |
| 675 | IPI00022744 | ISOFORM 1 OF EXPORTIN-2. |
| 676 | IPI00297550 | COAGULATION FACTOR XIII A CHAIN. |
| 677 | IPI00552419 | PROPIONYL-COA CARBOXYLASE ALPHA CHAIN, MITOCHONDRIAL ISOFORM C |
| | | PRECURSOR. |
| 678 | IPI00010154 | RAB GDP DISSOCIATION INHIBITOR ALPHA. |
| 679 | IPI00219673 | ISOFORM 1 OF GLUTATHIONE S-TRANSFERASE KAPPA 1. |
| 680 | IPI00027223 | ISOCITRATE DEHYDROGENASE [NADP] CYTOPLASMIC. |
| 681 | IPI00470674 | NADH-CYTOCHROME B5 REDUCTASE 1. |
| 682 | IPI00027192 | CDNA, FLJ79184, HIGHLY SIMILAR TO PROCOLLAGEN-LYSINE, 2- |
| | | OXOGLUTARATE 5-DIOXYGENASE 1. |
| 683 | IPI00003836 | UDP-GLUCURONOSYLTRANSFERASE 2B10. |
| 684 | IPI00377161 | ISOFORM 2 OF 3-HYDROXYISOBUTYRYL-COA HYDROLASE, MITOCHONDRIAL. |
| 685 | IPI00465431 | GALECTIN-3. |
| 686 | IPI00180675 | TUBULIN ALPHA-1A CHAIN. |
| 687 | IPI00013679 | 27 KDA PROTEIN. |
| 688 | IPI00025366 | CITRATE SYNTHASE, MITOCHONDRIAL. |
| 689 | IPI00329572 | PROTEIN KINASE C AND CASEIN KINASE SUBSTRATE IN NEURONS 3, ISOFORM |
| | | CRA_B. |
| 690 | IPI00465294 | CELL DIVISION CYCLE 5-LIKE PROTEIN. |
| 691 | IPI00029997 | 6-PHOSPHOGLUCONOLACTONASE. |
| 692 | IPI00221092 | 40S RIBOSOMAL PROTEIN S16. |
| 693 | IPI00479186 | ISOFORM M2 OF PYRUVATE KINASE ISOZYMES M1/M2. |
| 694 | IPI00006451 | VESICLE-FUSING ATPASE. |
| 695 | IPI00219018 | GLYCERALDEHYDE-3-PHOSPHATE DEHYDROGENASE. |
| 696 | IPI00329444 | ACYL-COENZYME A SYNTHETASE ACSM2B, MITOCHONDRIAL. |
| 697 | IPI00411937 | NUCLEOLAR PROTEIN 56. |
| 698 | IPI00026272 | HISTONE H2A TYPE 1-B/E. |
| 699 | IPI00007797 | FATTY ACID-BINDING PROTEIN, EPIDERMAL. |
| 700 | IPI00218606 | 40S RIBOSOMAL PROTEIN S23. |
| 701 | IPI00014053 | ISOFORM 1 OF MITOCHONDRIAL IMPORT RECEPTOR SUBUNIT TOM40 |
| | | HOMOLOG. |
| 702 | IPI00007133 | ISOFORM 3 OF CORDON-BLEU PROTEIN-LIKE 1. |
| 703 | IPI00011268 | CDNA FLJ77422, HIGHLY SIMILAR TO HOMO SAPIENS RNA BINDING PROTEIN, |
| | | AUTOANTIGENIC (HNRNP-ASSOCIATED WITH LETHAL YELLOW HOMOLOG |
| | | (MOUSE)), TRANSCRIPT VARIANT 1, MRNA (FRAGMENT). |
| 704 | IPI00026314 | ISOFORM 1 OF GELSOLIN. |
| 705 | IPI00220994 | CORE HISTONE MACRO-H2A.2 |
| 706 | IPI00299456 | FRUCTOSE-1,6-BISPHOSPHATASE ISOZYME 2. |
| 707 | IPI00016112 | ISOFORM 1 OF PEROXIDASIN HOMOLOG. |
| 708 | IPI00289758 | CALPAIN-2 CATALYTIC SUBUNIT. |
| 709 | IPI00013957 | MITOCHONDRIAL CARNITINE/ACYLCARNITINE CARRIER PROTEIN. |
| 710 | IPI00030275 | HEAT SHOCK PROTEIN 75 KDA, MITOCHONDRIAL. |
| 711 | IPI00003905 | SOLUTE CARRIER FAMILY 2, FACILITATED GLUCOSE TRANSPORTER MEMBER |
| | | 2. |
| 712 | IPI00013877 | ISOFORM 1 OF HETEROGENEOUS NUCLEAR RIBONUCLEOPROTEIN H3. |
| 713 | IPI00219365 | MOESIN. |
| 714 | IPI00215911 | DNA-(APURINIC OR APYRIMIDINIC SITE) LYASE. |
| 715 | IPI00293307 | PERILIPIN-2. |
| 716 | IPI00023086 | 39S RIBOSOMAL PROTEIN L15, MITOCHONDRIAL. |
| 717 | IPI00013809 | ISOFORM 1 OF MALEYLACETOACETATE ISOMERASE. |
| 718 | IPI00479145 | KERATIN, TYPE I CYTOSKELETAL 19. |
| 719 | IPI00007221 | PLASMA SERINE PROTEASE INHIBITOR. |
| 720 | IPI00028564 | INTERFERON-INDUCED GUANYLATE-BINDING PROTEIN 1. |
| 721 | IPI00012011 | COFILIN-1. |
| 722 | IPI00852768 | UNCHARACTERIZED PROTEIN. |
| 723 | IPI00007675 | CYTOPLASMIC DYNEIN 1 LIGHT INTERMEDIATE CHAIN 1. |
| 724 | IPI01008914 | EUKARYOTIC INITIATION FACTOR 4A-I ISOFORM 2. |
| 725 | IPI00019568 | PROTHROMBIN (FRAGMENT). |
| 726 | IPI00012048 | ISOFORM 1 OF NUCLEOSIDE DIPHOSPHATE KINASE A. |
| 727 | IPI00033349 | PROLACTIN REGULATORY ELEMENT-BINDING PROTEIN. |
| 728 | IPI00735641 | ISOFORM 1 OF 60 KDA LYSOPHOSPHOLIPASE. |
| 729 | IPI00644127 | ISOLEUCYL-TRNA SYNTHETASE, CYTOPLASMIC. |
| 730 | IPI00257508 | DIHYDROPYRIMIDINASE-RELATED PROTEIN 2. |
| 731 | IPI00000105 | MAJOR VAULT PROTEIN. |
| 732 | IPI00444262 | CDNA FLJ45706 FIS, CLONE FEBRA2028457, HIGHLY SIMILAR TO NUCLEOLIN. |
| 733 | IPI00024425 | UNCHARACTERIZED PROTEIN. |
| 734 | IPI00021263 | 14-3-3 PROTEIN ZETA/DELTA. |
| 735 | IPI00039626 | ISOFORM D OF CONSTITUTIVE COACTIVATOR OF PPAR-GAMMA-LIKE |
| | | PROTEIN 1. |
| 736 | IPI00014898 | ISOFORM 1 OF PLECTIN. |
| 737 | IPI00103483 | NEGATIVE ELONGATION FACTOR B. |
| 738 | IPI00401264 | ENDOPLASMIC RETICULUM RESIDENT PROTEIN 44. |
| 739 | IPI00218803 | ISOFORM B OF FIBULIN-1. |
| 740 | IPI00333619 | ISOFORM 1 OF FATTY ALDEHYDE DEHYDROGENASE. |
| 741 | IPI00884105 | LYSOSOME-ASSOCIATED MEMBRANE GLYCOPROTEIN 1. |
| 742 | IPI00026781 | FATTY ACID SYNTHASE. |
| 743 | IPI01014863 | ACETYL-COA ACETYLTRANSFERASE, CYTOSOLIC. |
| 744 | IPI00375577 | TRANSMEMBRANE PROTEIN 65. |
| 745 | IPI00062206 | UNCHARACTERIZED PROTEIN. |
| 746 | IPI00021812 | NEUROBLAST DIFFERENTIATION-ASSOCIATED PROTEIN AHNAK. |
| 747 | IPI00022002 | CDNA FLJ54536, HIGHLY SIMILAR TO MITOCHONDRIAL 28S RIBOSOMAL |
| | | PROTEIN S27. |
| 748 | IPI00465085 | ISOFORM 1 OF NICOTINATE PHOSPHORIBOSYLTRANSFERASE. |
| 749 | IPI00019599 | ISOFORM 1 OF UBIQUITIN-CONJUGATING ENZYME E2 VARIANT 1. |
| 750 | IPI00375631 | UBIQUITIN-LIKE PROTEIN ISG15. |
| 751 | IPI00032304 | PLASTIN-1. |
| 752 | IPI00023748 | NASCENT POLYPEPTIDE-ASSOCIATED COMPLEX SUBUNIT ALPHA. |
| 753 | IPI00018342 | ADENYLATE KINASE ISOENZYME 1. |
| 754 | IPI00020906 | INOSITOL MONOPHOSPHATASE 1. |
| 755 | IPI00005809 | SERUM DEPRIVATION-RESPONSE PROTEIN. |
| 756 | IPI00448751 | ISOFORM 3 OF SHOOTIN-1. |
| 757 | IPI00006034 | CYSTEINE-RICH PROTEIN 2. |
| 758 | IPI00042580 | ISOFORM 1 OF APOLIPOPROTEIN O. |
| 759 | IPI00221234 | ISOFORM 1 OF ALPHA-AMINOADIPIC SEMIALDEHYDE DEHYDROGENASE. |
| 760 | IPI00784614 | SEPTIN-9 ISOFORM A. |
| 761 | IPI00022254 | ISOFORM 1 OF UBIQUITIN-LIKE-CONJUGATING ENZYME ATG3. |
| 762 | IPI00553177 | ISOFORM 1 OF ALPHA-1-ANTITRYPSIN. |
| 763 | IPI00658013 | NUCLEOPHOSMIN ISOFORM 3. |
| 764 | IPI00219518 | ADP-RIBOSYLATION FACTOR-LIKE PROTEIN 1. |
| 765 | IPI00013297 | 28 KDA HEAT- AND ACID-STABLE PHOSPHOPROTEIN. |
| 766 | IPI00009747 | LANOSTEROL SYNTHASE. |
| 767 | IPI00030229 | UNCHARACTERIZED PROTEIN. |
| 768 | IPI00218591 | ISOFORM ASF-2 OF SERINE/ARGININE-RICH SPLICING FACTOR 1. |
| 769 | IPI00031091 | EF-HAND DOMAIN-CONTAINING PROTEIN D1. |
| 770 | IPI00021338 | DIHYDROLIPOYLLYSINE-RESIDUE ACETYLTRANSFERASE COMPONENT OF |
| | | PYRUVATE DEHYDROGENASE COMPLEX, MITOCHONDRIAL. |
| 771 | IPI00215780 | 40S RIBOSOMAL PROTEIN S19. |
| 772 | IPI00099595 | 17-BETA-HYDROXYSTEROID DEHYDROGENASE TYPE 6. |
| 773 | IPI00017342 | RHO-RELATED GTP-BINDING PROTEIN RHOG. |
| 774 | IPI00643527 | ISOFORM 2 OF PHOSPHOINOSITIDE 3-KINASE ADAPTER PROTEIN 1. |
| 775 | IPI00916765 | CDNA FLJ53959, HIGHLY SIMILAR TO MITOCHONDRIAL INNER MEMBRANE |
| | | PROTEIN. |
| 776 | IPI00411639 | LAMININ RECEPTOR-LIKE PROTEIN LAMRL5. |
| 777 | IPI00015953 | ISOFORM 1 OF NUCLEOLAR RNA HELICASE 2. |
| 778 | IPI00017855 | ACONITATE HYDRATASE, MITOCHONDRIAL. |
| 779 | IPI00004902 | ISOFORM 1 OF ELECTRON TRANSFER FLAVOPROTEIN SUBUNIT BETA. |
| 780 | IPI00554811 | ACTIN-RELATED PROTEIN 2/3 COMPLEX SUBUNIT 4. |
| 781 | IPI00011075 | ALANINE--GLYOXYLATE AMINOTRANSFERASE 2, MITOCHONDRIAL. |
| 782 | IPI00029307 | HISTAMINE N-METHYLTRANSFERASE ISOFORM 2. |
| 783 | IPI00019755 | GLUTATHIONE S-TRANSFERASE OMEGA-1. |
| 784 | IPI00002370 | LEUKOTRIENE-B(4) OMEGA-HYDROXYLASE 2. |
| 785 | IPI00418262 | FRUCTOSE-BISPHOSPHATE ALDOLASE. |
| 786 | IPI00028091 | ACTIN-RELATED PROTEIN 3. |
| 787 | IPI00021891 | ISOFORM GAMMA-B OF FIBRINOGEN GAMMA CHAIN. |
| 788 | IPI00465038 | ISOFORM 2 OF FIBULIN-2. |
| 789 | IPI00031420 | UDP-GLUCOSE 6-DEHYDROGENASE. |
| 790 | IPI00003925 | ISOFORM 1 OF PYRUVATE DEHYDROGENASE El COMPONENT SUBUNIT |
| | | BETA, MITOCHONDRIAL. |
| 791 | IPI00418471 | VIMENTIN. |
| 792 | IPI00029019 | ISOFORM 2 OF UBIQUITIN-ASSOCIATED PROTEIN 2-LIKE. |
| 793 | IPI00641582 | BAG FAMILY MOLECULAR CHAPERONE REGULATOR 3. |
| 794 | IPI00020436 | RAS-RELATED PROTEIN RAB-11B. |
| 795 | IPI00096066 | SUCCINYL-COA LIGASE [GDP-FORMING] SUBUNIT BETA, MITOCHONDRIAL. |
| 796 | IPI00329495 | ISOFORM 1 OF ACTIN-BINDING LIM PROTEIN 1. |
| 797 | IPI00008494 | INTERCELLULAR ADHESION MOLECULE 1. |
| 798 | IPI00889534 | CARBAMOYL-PHOSPHATE SYNTHASE [AMMONIA], MITOCHONDRIAL |
| | | ISOFORM A PRECURSOR. |
| 799 | IPI00294178 | ISOFORM 1 OF SERINE/THREONINE-PROTEIN PHOSPHATASE 2A 65 KDA |
| | | REGULATORY SUBUNIT A BETA ISOFORM. |
| 800 | IPI00168603 | CHOLINE DEHYDROGENASE, MITOCHONDRIAL. |
| 801 | IPI00002491 | ISOFORM 9 OF SORBIN AND SH3 DOMAIN-CONTAINING PROTEIN 1. |
| 802 | IPI00008530 | 60S ACIDIC RIBOSOMAL PROTEIN P0. |
| 803 | IPI00038356 | ISOFORM 3 OF ARGINASE-1. |
| 804 | IPI00413344 | COFILIN-2. |
| 805 | IPI00640703 | EXPORTIN-5. |
| 806 | IPI00008274 | ISOFORM 1 OF ADENYLYL CYCLASE-ASSOCIATED PROTEIN 1. |
| 807 | IPI00293665 | KERATIN, TYPE II CYTOSKELETAL 6B. |
| 808 | IPI00032179 | ANTITHROMBIN-III. |
| 809 | IPI00306516 | MITOCHONDRIAL IMPORT INNER MEMBRANE TRANSLOCASE SUBUNIT |
| | | TIM44. |
| 810 | IPI00033494 | MYOSIN REGULATORY LIGHT CHAIN 12B. |
| 811 | IPI00018260 | ARMADILLO REPEAT-CONTAINING PROTEIN 1. |
| 812 | IPI00059366 | ISOFORM 3 OF CORE HISTONE MACRO-H2A.1 |
| 813 | IPI00384495 | ISOFORM 3 OF E3 UBIQUITIN-PROTEIN LIGASE NEDD4. |
| 814 | IPI00306369 | TRNA (CYTOSINE(34)-C(5))-METHYLTRANSFERASE. |
| 815 | IPI00291578 | ISOFORM 1 OF PHOSPHORIBOSYL PYROPHOSPHATE SYNTHASE-ASSOCIATED |
| | | PROTEIN 1. |
| 816 | IPI00026185 | ISOFORM 1 OF F-ACTIN-CAPPING PROTEIN SUBUNIT BETA. |
| 817 | IPI00181893 | ISOFORM 4 OF PHOSPHORYLASE B KINASE REGULATORY SUBUNIT BETA. |
| 818 | IPI00297261 | TYROSINE-PROTEIN PHOSPHATASE NON-RECEPTOR TYPE 1. |
| 819 | IPI00029772 | DIHYDROPYRIMIDINE DEHYDROGENASE [NADP+]. |
| 820 | IPI00027497 | GLUCOSE-6-PHOSPHATE ISOMERASE. |
| 821 | IPI00026602 | HLA CLASS I HISTOCOMPATIBILITY ANTIGEN, B-41 ALPHA CHAIN. |
| 822 | IPI00384401 | MYOSIN-REACTIVE IMMUNOGLOBULIN KAPPA CHAIN VARIABLE REGION |
| | | (FRAGMENT). |
| 823 | IPI00065063 | DEHYDROGENASE/REDUCTASE SDR FAMILY MEMBER 1. |
| 824 | IPI00470470 | ISOFORM 2 OF PROBABLE ARYLFORMAMIDASE. |
| 825 | IPI00294186 | ISOFORM 1 OF SERINE BETA-LACTAMASE-LIKE PROTEIN LACTB, |
| | | MITOCHONDRIAL. |
| 826 | IPI00018783 | INOSINE TRIPHOSPHATE PYROPHOSPHATASE. |
| 827 | IPI00100775 | ISOFORM 1 OF UPF0366 PROTEIN C11ORF67. |
| 828 | IPI00967700 | UNCHARACTERIZED PROTEIN. |
| 829 | IPI00019353 | ISOFORM 1 OF ACYLGLYCEROL KINASE, MITOCHONDRIAL. |
| 830 | IPI00010274 | TPSAB1 PROTEIN. |
| 831 | IPI00024462 | DIHYDROOROTATE DEHYDROGENASE (QUINONE), MITOCHONDRIAL. |
| 832 | IPI00783987 | COMPLEMENT C3 (FRAGMENT). |
| 833 | IPI00005563 | ISOFORM 1 OF TUBULOINTERSTITIAL NEPHRITIS ANTIGEN-LIKE. |
| 834 | IPI00304612 | 60S RIBOSOMAL PROTEIN L13A. |
| 835 | IPI00289334 | ISOFORM 1 OF FILAMIN-B. |
| 836 | IPI00037283 | ISOFORM 5 OF DYNAMIN-1-LIKE PROTEIN. |
| 837 | IPI00924816 | MYOTROPHIN. |
| 838 | IPI00218319 | ISOFORM 2 OF TROPOMYOSIN ALPHA-3 CHAIN. |
| 839 | IPI00014850 | ASTROCYTIC PHOSPHOPROTEIN PEA-15. |
| 840 | IPI00221088 | 40S RIBOSOMAL PROTEIN S9. |
| 841 | IPI00292150 | LATENT-TRANSFORMING GROWTH FACTOR BETA-BINDING PROTEIN 2. |
| 842 | IPI00017375 | PROTEIN TRANSPORT PROTEIN SEC23A. |
| 843 | IPI00022420 | RETINOL-BINDING PROTEIN 4. |
| 844 | IPI00000877 | HYPDXIA UP-REGULATED PROTEIN 1. |
| 845 | IPI00024317 | ISOFORM LONG OF GLUTARYL-COA DEHYDROGENASE, MITOCHONDRIAL. |
| 846 | IPI00337541 | NAD(P) TRANSHYDROGENASE, MITOCHONDRIAL. |
| 847 | IPI00000684 | ISOFORM AGX2 OF UDP-N-ACETYLHEXOSAMINE PYROPHOSPHORYLASE. |
| 848 | IPI00386533 | ISOFORM E OF EUKARYOTIC TRANSLATION INITIATION FACTOR 4 GAMMA 1. |
| 849 | IPI00007814 | V-TYPE PROTON ATPASE SUBUNIT C 1. |
| 850 | IPI00020416 | TRIPEPTIDYL-PEPTIDASE 2. |
| 851 | IPI00033217 | ALPHA-AMINOADIPIC SEMIALDEHYDE SYNTHASE, MITOCHONDRIAL. |
| 852 | IPI00029111 | DIHYDROPYRIMIDINASE-RELATED PROTEIN 3 ISOFORM 1. |
| 853 | IPI00448925 | 44 KDA PROTEIN. |
| 854 | IPI00794316 | 24 KDA PROTEIN. |
| 855 | IPI00008178 | ISOFORM 1 OF SODIUM/POTASSIUM-TRANSPORTING ATPASE SUBUNIT |
| | | GAMMA. |
| 856 | IPI00219782 | RETINOL-BINDING PROTEIN 5. |
| 857 | IPI00396131 | CDNA FLJ56221, HIGHLY SIMILAR TO YTH DOMAIN PROTEIN 3. |
| 858 | IPI00975939 | SAA2-SAA2 PROTEIN. |
| 859 | IPI00218918 | ANNEXIN A1. |
| 860 | IPI00001676 | ISOFORM 2 OF NUCLEAR PROTEIN LOCALIZATION PROTEIN 4 HOMOLOG. |
| 861 | IPI00001159 | TRANSLATIONAL ACTIVATOR GCN1. |
| 862 | IPI00013195 | 39S RIBOSOMAL PROTEIN L49, MITOCHONDRIAL. |
| 863 | IPI00003815 | RHO GDP-DISSOCIATION INHIBITOR 1. |
| 864 | IPI00218236 | SERINE/THREONINE-PROTEIN PHOSPHATASE PP1-BETA CATALYTIC SUBUNIT. |
| 865 | IPI00006592 | ISOFORM 1 OF MITOCHONDRIAL PEPTIDE METHIONINE SULFOXIDE |
| | | REDUCTASE. |
| 866 | IPI00158296 | UNCHARACTERIZED PROTEIN. |
| 867 | IPI00009949 | PROTEASOME INHIBITOR PI31 SUBUNIT. |
| 868 | IPI00016910 | EUKARYOTIC TRANSLATION INITIATION FACTOR 3 SUBUNIT C. |
| 869 | IPI00005578 | EH DOMAIN-CONTAINING PROTEIN 4. |
| 870 | IPI00299048 | ISOFORM 1 OF RAS GTPASE-ACTIVATING-LIKE PROTEIN IQGAP2. |
| 871 | IPI00026958 | ISOFORM SHORT OF NADPH:ADRENODOXIN OXIDOREDUCTASE, |
| | | MITOCHONDRIAL. |
| 872 | IPI00291262 | ISOFORM 1 OF CLUSTERIN. |
| 873 | IPI00218192 | ISOFORM 2 OF INTER-ALPHA-TRYPSIN INHIBITOR HEAVY CHAIN H4. |
| 874 | IPI00954463 | KERATIN, TYPE II CYTOSKELETAL 7. |
| 875 | IPI00298497 | FIBRINOGEN BETA CHAIN. |
| 876 | IPI00396321 | LEUCINE-RICH REPEAT-CONTAINING PROTEIN 59. |
| 877 | IPI00015285 | ETHANOLAMINE-PHOSPHATE CYTIDYLYLTRANSFERASE. |
| 878 | IPI00293464 | DNA DAMAGE-BINDING PROTEIN 1. |
| 879 | IPI00025273 | ISOFORM LONG OF TRIFUNCTIONAL PURINE BIOSYNTHETIC PROTEIN |
| | | ADENOSINE-3. |
| 880 | IPI00216219 | ISOFORM LONG OF TIGHT JUNCTION PROTEIN ZO-1. |
| 881 | IPI00339225 | ISOFORM 5 OF FIBRONECTIN. |
| 882 | IPI00011416 | DELTA(3,5)-DELTA(2,4)-DIENOYL-COA ISOMERASE, MITOCHONDRIAL. |
| 883 | IPI00013323 | CYTOCHROME P450 2C19. |
| 884 | IPI00003944 | LIPOAMIDE ACYLTRANSFERASE COMPONENT OF BRANCHED-CHAIN ALPHA- |
| | | KETO ACID DEHYDROGENASE COMPLEX, MITOCHONDRIAL. |
| 885 | IPI00010720 | T-COMPLEX PROTEIN 1 SUBUNIT EPSILON. |
| 886 | IPI00009030 | ISOFORM LAMP-2A OF LYSOSOME-ASSOCIATED MEMBRANE GLYCOPROTEIN |
| | | 2. |
| 887 | IPI00554788 | KERATIN, TYPE I CYTOSKELETAL 18. |
| 888 | IPI00065500 | BRO1 DOMAIN-CONTAINING PROTEIN BROX. |
| 889 | IPI00014363 | BETAINE--HOMOCYSTEINE S-METHYLTRANSFERASE 2. |
| 890 | IPI00745872 | ISOFORM 1 OF SERUM ALBUMIN. |
| 891 | IPI00465315 | CYTOCHROME C. |
| 892 | IPI00549413 | UNCHARACTERIZED PROTEIN. |
| 893 | IPI00550991 | ISOFORM 2 OF ALPHA-1-ANTICHYMOTRYPSIN. |
| 894 | IPI00024266 | MICROSOMAL GLUTATHIONE S-TRANSFERASE 3. |
| 895 | IPI00292946 | THYROXINE-BINDING GLOBULIN. |
| 896 | IPI00013698 | N-ACYLSPHINGOSINE AMIDOHYDROLASE (ACID CERAMIDASE) 1, ISOFORM |
| | | CRA_C. |
| 897 | IPI00217561 | ISOFORM BETA-1C OF INTEGRIN BETA-1. |
| 898 | IPI00022426 | PROTEIN AMBP. |
| 899 | IPI00013193 | ISOFORM 1 OF MYOSIN-VIIA. |
| 900 | IPI00217296 | ISOFORM 3 OF SERINE/THREONINE-PROTEIN PHOSPHATASE 2A ACTIVATOR. |
| 901 | IPI00021370 | ISOFORM 1 OF UBIQUITIN-CONJUGATING ENZYME E2 K. |
| 902 | IPI00018314 | SEC14-LIKE PROTEIN 2. |
| 903 | IPI00479058 | 40S RIBOSOMAL PROTEIN S15. |
| 904 | IPI00329598 | ESTRADIOL 17-BETA-DEHYDROGENASE 11. |
| 905 | IPI00024787 | VERY LONG-CHAIN ACYL-COA SYNTHETASE. |
| 906 | IPI00790342 | 60S RIBOSOMAL PROTEIN L6. |
| 907 | IPI00021440 | ACTIN, CYTOPLASMIC 2. |
| 908 | IPI01011912 | PHOSPHOGLYCERATE KINASE. |
| 909 | IPI00005668 | ALDO-KETO REDUCTASE FAMILY 1 MEMBER C2. |
| 910 | IPI00017726 | ISOFORM 1 OF 3-HYDROXYACYL-COA DEHYDROGENASE TYPE-2. |
| 911 | IPI00021304 | KERATIN, TYPE II CYTOSKELETAL 2 EPIDERMAL. |
| 912 | IPI00024911 | ENDOPLASMIC RETICULUM RESIDENT PROTEIN 29. |
| 913 | IPI00028006 | PROTEASOME SUBUNIT BETA TYPE-2. |
| 914 | IPI00029733 | ALDO-KETO REDUCTASE FAMILY 1 MEMBER Cl. |
| 915 | IPI00171257 | SERINE/THREONINE-PROTEIN KINASE R101 ISOFORM 2. |
| 916 | IPI00293350 | TRANSLIN-ASSOCIATED PROTEIN X. |
| 917 | IPI00297779 | T-COMPLEX PROTEIN 1 SUBUNIT BETA. |
| 918 | IPI00298176 | GLUTATHIONE PEROXIDASE 2. |
| 919 | IPI00397498 | ISOFORM 2 OF CARBAMOYL-PHOSPHATE SYNTHASE [AMMONIA], |
| | | MITOCHONDRIAL. |
| 920 | IPI00465248 | ISOFORM ALPHA-ENOLASE OF ALPHA-ENOLASE. |
| 921 | IPI00514814 | ALDO-KETO REDUCTASE FAMILY 1, MEMBER Cl (DIHYDRODIOL |
| | | DEHYDROGENASE 1. |
| 922 | IPI00607693 | LIVER CARBOXYLESTERASE 1 ISOFORM C PRECURSOR. |
| 923 | IPI00643595 | UNCHARACTERIZED PROTEIN. |
| 924 | IPI00643908 | CDNA FLJ53700, HIGHLY SIMILAR TO HEPATOMA-DERIVED GROWTH |
| | | FACTOR. |
| 925 | IPI00789078 | 14 KDA PROTEIN. |
| 926 | IPI00789134 | GLYCERALDEHYDE-3-PHOSPHATE DEHYDROGENASE. |
| 927 | IPI00792207 | ALDEHYDE DEHYDROGENASE, MITOCHONDRIAL ISOFORM 2 PRECURSOR. |
| 928 | IPI00794900 | CDNA FLJ56016, HIGHLY SIMILAR TO C-1-TETRAHYDROFOLATE SYNTHASE, |
| | | CYTOPLASMIC. |
| 929 | IPI00871858 | CYTOCHROME B-5 ISOFORM 1 VARIANT. |
| 930 | IPI00871915 | UNCHARACTERIZED PROTEIN. |
| 931 | IPI00878282 | 23 KDA PROTEIN. |
| 932 | IPI00927949 | ISOFORM 1 OF ALCOHOL DEHYDROGENASE 4. |
| 933 | IPI00936678 | HYPOTHETICAL PROTEIN LOC100288568. |
| 934 | IPI00940264 | GALACTOKINASE. |
| 935 | IPI00942507 | CDNA FLJ57637, HIGHLY SIMILAR TO LIVER CARBOXYLESTERASE 1. |
| 936 | IPI00942659 | 14 KDA PROTEIN. |
| 937 | IPI00945760 | HYDROXYMETHYLGLUTARYL-COA SYNTHASE, MITOCHONDRIAL ISOFORM 2 |
| | | PRECURSOR. |
| 938 | IPI00978288 | PROTEASOME SUBUNIT BETA TYPE-2 ISOFORM 2. |
| 939 | IPI00980553 | CDNA FLJ50204, HIGHLY SIMILAR TO 2,4-DIENOYL-COA REDUCTASE, |
| | | MITOCHONDRIAL. |
| 940 | IPI00981251 | UNCHARACTERIZED PROTEIN. |
| 941 | IPI01009477 | UNCHARACTERIZED PROTEIN. |
| 942 | IPI00872991 | UNCHARACTERIZED PROTEIN. |
| 943 | IPI01011174 | CDNA, FLJ79393, HIGHLY SIMILAR TO SARCOSINE DEHYDROGENASE, |
| | | MITOCHONDRIAL. |
| 944 | IPI01012004 | CDNA PSEC0175 FIS, CLONE OVARC1000169, HIGHLY SIMILAR TO PROTEIN |
| | | DISULFIDE-ISOMERASE A3. |
| 945 | IPI01012473 | CDNA FLJ57418, HIGHLY SIMILAR TO SHORT/BRANCHED CHAIN SPECIFIC |
| | | ACYL- COADEHYDROGENASE, MITOCHONDRIAL. |
| 946 | IPI01012766 | CDNA, FLJ79260, HIGHLY SIMILAR TO ACTIN, CYTOPLASMIC 2. |
| 947 | IPI00745233 | GLUTATHIONE S-TRANSFERASE A2. |
| 948 | IPI01014091 | UNCHARACTERIZED PROTEIN. |
| 949 | IPI01014382 | UNCHARACTERIZED PROTEIN. |
| 950 | IPI01015455 | 166 KDA PROTEIN. |
| 951 | IPI01015522 | CDNA FLJ55253, HIGHLY SIMILAR TO ACTIN, CYTOPLASMIC 1. |
| 952 | IPI00000874 | PEROXIREDOXIN-1. |
| 953 | IPI00007752 | TUBULIN BETA-2C CHAIN. |
| 954 | IPI00010779 | ISOFORM 1 OF TROPOMYOSIN ALPHA-4 CHAIN. |
| 955 | IPI00013894 | STRESS-INDUCED-PHOSPHOPROTEIN 1. |
| 956 | IPI00021439 | ACTIN, CYTOPLASMIC 1. |
| 957 | IPI00022774 | TRANSITIONAL ENDOPLASMIC RETICULUM ATPASE. |
| 958 | IPI00022895 | ISOFORM 1 OF ALPHA-1B-GLYCOPROTEIN. |
| 959 | IPI00037070 | UNCHARACTERIZED PROTEIN. |
| 960 | IPI00062003 | ACAT1 PROTEIN. |
| 961 | IPI00514320 | UNCHARACTERIZED PROTEIN. |
| 962 | IPI00604607 | UNCHARACTERIZED PROTEIN. |
| 963 | IPI00645363 | PUTATIVE UNCHARACTERIZED PROTEIN DKFZP686P15220. |
| 964 | IPI00646055 | UNCHARACTERIZED PROTEIN. |
| 965 | IPI00654755 | HEMOGLOBIN SUBUNIT BETA. |
| 966 | IPI00790739 | UNCHARACTERIZED PROTEIN. |
| 967 | IPI00792290 | ISOFORM 2 OF WD REPEAT-CONTAINING PROTEIN 66. |
| 968 | IPI00792677 | CDNA FLJ60097, HIGHLY SIMILAR TO TUBULIN ALPHA-UBIQUITOUS CHAIN. |
| 969 | IPI00845339 | CDNA FLJ54370, HIGHLY SIMILAR TO HEAT SHOCK 70 KDA PROTEIN 1. |
| 970 | IPI00903145 | RADIXIN. |
| 971 | IPI00917331 | UNCHARACTERIZED PROTEIN. |
| 972 | IPI00925411 | UNCHARACTERIZED PROTEIN. |
| 973 | IPI00925747 | PEPTIDYL-PROLYL CIS-TRANS ISOMERASE. |
| 974 | IPI00927101 | UNCHARACTERIZED PROTEIN. |
| 975 | IPI00930124 | PUTATIVE UNCHARACTERIZED PROTEIN DKFZP686C11235. |
| 976 | IPI00783987 | COMPLEMENT C3 (FRAGMENT). |
| 977 | IPI01011434 | UNCHARACTERIZED PROTEIN. |
| 978 | IPI01012499 | UNCHARACTERIZED PROTEIN. |
| 979 | IPI00746165 | ISOFORM 1 OF WD REPEAT-CONTAINING PROTEIN 1 |
| 980 | O00418 | Eucaryotic elongation factor 2 kinase |
| 981 | Q13043 | serine/threonine kinase 4 |
| 982 | Q13188 | serine/threonine kinase 3 (STE20 homolog) |
| 983 | Q9BXU1 | serine/threonine kinase 31 |

1. A method for identifying biomarkers specific for a particular disease comprising the steps
a) determining if a particular protein is differentially expressed in cause of this particular disease by 2-D gel electrophoresis and
b) determining if this particular protein is differentially expressed in cause of this particular disease by liquid chromatography-mass spectrometry (LC-MS).

2. A method according to claim 1, wherein the gel-based approach is 2D-DIGE and wherein the LC-MS-based approach is MALDI, preferably MALDI-TOF-MS or nan-HPLC-ESI-MS/MS.

3. A method according to one of the previous claims, wherein the particular disease is hepatocellular carcinoma (HCC).

4. Biomarker for HCC identified by a method according to one of the previous claims and selected from the proteins defined by SEQ ID No. 1 to 983, the respective homologues of SEQ ID No. 1 to 983 with at least 95% identity in amino acid sequence, the respective isoforms of proteins defined by SEQ ID No. 1 to 983, the respective partial sequences of SEQ ID No. 1 to 983.

5. Biomarker according to claim 4, characterized in that the biomarker is selected from PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31.

6. Use of one or more proteins selected from the proteins defined by SEQ ID No. 1 to 983, the respective homologues of SEQ ID No. 1 to 983 with at least 95% identity in amino acid sequence, the respective isoforms of proteins defined by SEQ ID No. 1 to 983, the respective partial sequences of SEQ ID No. 1 to 983 as biomarker(s) for HCC.

7. Use as claimed in claim 6, wherein the protein(s) is/are selected from PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31.

8. Use according to claim 6 or 7 for differential diagnosis, in particular for early recognition, diagnosis, prognosis, evaluation of disease progression, prediction of outcome, evaluation of treatment, surveillance of treatment, surveillance of after-treatment of HCC.

9. A method for studying a biological sample for HCC, wherein the sample is studied for one or more biomarker(s) for HCC and wherein the biomarker(s) is/are differentially expressed in relation to the healthy state indicating the presence of HCC, characterized in that the biomarker(s) is/are selected from the group comprising proteins defined by SEQ ID No. 1 to 983, the respective isoforms of the proteins defined by SEQ ID. No. 1 to 983, the respective homologues of SEQ ID No. 1 to 983 with at least 95% identity in amino acid sequence, the respective partial sequences of SEQ ID No. 1 to 983.

10. A method according to claim 9, characterized in that the biomarker(s) is/are selected from the group comprising PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4 Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31.

11. A method according to claim 9 or 10, wherein the sample is a human sample.

12. A method according to claims 9 to 11, wherein the sample is blood serum, blood plasma, whole blood, a biopsy sample, in particular a liver biopsy sample.

13. Diagnostic device or test kit for analysing the amount of at least one biomarker selected from the group comprising proteins defined by SEQ ID No. 1 to 983, preferably selected from proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences, and wherein the diagnostic device or test kit comprises detection reagents and further aids.

14. A diagnostic device or a test kit according to claim 13, wherein the detection reagent comprises an antibody specific for the respective biomarker.

15. Use of a method as claimed in claims 9 to 12 to identify in a sample at least one HCC specific biomarker as defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences.

16. Use of at least one specific biomarker selected from the group of defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences for screening pharmaceutical compounds for HCC.

17. Screening assay for the identification and validation of pharmaceutical compounds comprising one or more of biomarkers for HCC selected from the group comprising proteins defined by SEQ ID No. 1 to 983, preferably proteins PPA1, IGHG1, IGHV4-31, SERPINA1, VIM, LMNA, KRT18, GAPDH, PKM2, HSPA9, HSPA5, TRAP1, ACO2, HSPA8, CCT5, ECH1, SOD1, CA2, QDPR, AGXT, SORD, GLUD1, CPS1, ALDH6A1, GRHPR, UGP2, ALDH2, ECHS1, AKR1C4, ALDH1A1, MPST, ASS1, ACADS, ALDOB, ACAADSB, KHK, SARDH, FTCD, CES1, BDH1, PBLD, FBP1, BHMT, GNMT, ALB, PPIA, MTHFD1, ACAT1, PCK2, GATM, ADH1B, ADH4, Elongation factor 2 (eEF2), Elongation factor 2 kinase, Isoform of 14-3-3 Protein Sigma, Serine/Threonine Kinase 3, Serine/Threonine Kinase 4, Serine/Threonine Kinase 31 and the respective isoforms, the respective homologues with at least 95% identity in amino acid sequence, the respective partial sequences, and wherein the screening assay comprises detection reagents and further aids.
